(19) United States
(12) Patent Application Publication
(10) Pub. No.: US 2003/0083716 A1
(43) Pub. Date: May 1, 2003

(54) Intelligent brain pacemaker for real-time monitoring and controlling of epileptic seizures

(76) Inventors: Miguel A.L. Nicolelis, Chapel Hill, NC (US); Erika E. Fanselow, Providence, RI (US); Ashlan P. Reid, Philadelphia, PA (US)

Correspondence Address: JENKINS & WILSON, PA, 3100 TOWER BLVD, SUITE 1400, DURHAM, NC, 27707, US

(21) Appl. No.: 10/004,457

(22) Filed: Oct. 23, 2001

(51) Int. Cl. A61N 1/18

(52) U.S. Cl. 607/45

(57) ABSTRACT

An intelligent brain pace maker for detecting and ameliorating epileptic seizures is disclosed. A method for detecting and ameliorating epileptic seizures is also disclosed. Further, a method of increasing the time interval between epileptic seizures is disclosed.

### TECHNICAL FIELD

The present invention relates generally to detection and amelioration of oncoming epileptic seizures and more specifically to amelioration of epileptic seizures by stimulation of the trigeminal cranial nerve.

Abbreviations

| | |
|---|---|
| ANOVA | analysis of variance |
| ASD | automatic seizure detector |
| EEG | electroencephalogram |
| EKG | electrocardiogram |
| EPSP | excitatory postsynaptic potential |
| IO | infraorbital |
| MANOVA | multivariate analysis of variance |
| NTS | nucleus of the solitary tract |
| PTZ | pentylenetetrazole |
| SI | primary somatosensory cortices |
| TTL | transistor-transistor logic |
| VNS | vagus nerve stimulation |
| VPM | ventral posterior medial thalamus |

### BACKGROUND ART

Epileptic seizures are the outward manifestation of excessive and/or hypersynchronous abnormal activity of neurons in the cerebral cortex. Seizures are usually self-limiting. Many types of seizures occur. The behavioral features of a seizure reflect the function of the portion of the cortex where the hyperactivity is occurring. Seizures can be generalized, appearing to involve the entire brain simultaneously. Generalized seizures can result in the loss of conscious awareness only and are then called absence seizures (commonly referred to as “petit mal”). Alternatively, the generalized seizure may result in a convulsion with tonic-clonic contractions of the muscles (“grand mal” seizure). Some types of seizures, partial seizures, begin in one part of the brain and remain local. The person may remain conscious throughout the seizure. If the person loses consciousness the seizure is referred to as a complex partial seizure.

Seminal neurophysiological studies performed several decades ago demonstrated that stimulation of either cranial nerves or areas of the brainstem can cause desynchronization of the cortical EEG (Moruzzi & Magoun, (1949) Electroencephalogr. Clin. Neurophysiol. 1: 455-473; Zanchetti et al., (1952) Electroencephalogr. Clin. Neurophysiol. 4: 357-361; Magnes et al., (1 961) Arch. Ital. Biol. 99: 33-67; Chase et al., (1967) Brain Res. 5: 236-249). Such desynchronization typically reflects a state of arousal and full vigilance in mammals and is opposite to the high degree of EEG synchronization observed during seizure activity. Building on these classical findings, several researchers showed that stimulation of the vagus nerve can lead to EEG desynchronization (Zanchetti et al., (1952) Electroencephalogr. Clin. Neurophysiol. 4: 357-361; Chase et al., (1966) Exp. Neurol. 16: 36-49; Chase et al., (1967) Brain Res. 5: 236-249; Chase & Nakamura, (1968) Brain Res. 5: 236-249). More recently, several studies have demonstrated that the desynchronization induced by vagus nerve stimulation (VNS) in dogs can be used to reduce strychnine or pentylenetetrazole (PTZ)-induced seizure activity (Zabara, (1985), Epilepsia 26: 518; Zabara (1992) Epilepsia 33:1005-1012). This paradigm was demonstrated subsequently to be effective in other animals, with other seizure models (Lockard et al., (1990) Epilepsia 31[Suppl 2]: S20-S26; Woodbury & Woodbury, (1990) Epilepsia 31[Suppl 2]: S7-S19; McLachlan, (1993) Epilepsia 34: 918-923), and has been used with moderate success in treating humans who have otherwise intractable epileptic seizures (Penry & Dean, (1990) Epilepsia 31 [Suppl. 2]: S40-S43; Uthman et al., (1990) Epilepsia 31[Suppl. 2]: S44-S50; Uthman et al., (1993) Neurology 43: 1338-1345; Ben-Menachem et al., (1994) Epilepsia 35: 616-626; Vagus Nerve Stimulation Study Group, (1995) Neurology45: 224-230; McLachlan, (1997) J. Clin. Neurophysiol. 14: 358-368; Schachter,& Saper, (1998) Epilepsia 39: 677-686). Because 0.5-2% of the population has epilepsy (Schachter & Saper, (1998) Epilepsia 39: 677-686; McNamara, (1999) Nature 399[Suppl. 6738]: A15-A22), since ten to fifty percent of these patients do not respond well to antiepileptic medications and/or may not be candidates for resective epilepsy surgery (McLachlan, (1997) J. Clin. Neurophysiol. 14: 358-368; Schachter & Saper, (1998) Epilepsia 39: 677-686), there is a substantial need for potential alternative therapies for chronic seizures. Indeed, the VNS technique has recently received FDA approval and is currently being employed in human patients.

There are, however, a number of limiting factors of the VNS technique, which, if addressed, would greatly increase the efficacy and applicability of cranial nerve stimulation for seizure reduction in patients. First, the standard implementation of VNS in humans typically involves stimulating the vagus nerve on a fixed, intermittent duty cycle (e.g., 30 seconds on; 5 minutes off; 24 hours a day), independently of whether any seizure activity is ongoing or imminent (although the use of manual patient- or caregiver-triggered stimulation via a handheld magnet has also been used) (Terry et al., (1990) Epilepsia 31 [Suppl 2]: S33-S37; Uthman et al., (1993) Neurology 43: 1338-1345). This type of protocol has been used in previous studies for two main reasons. First, although continuous stimulation may have a greater therapeutic effect than intermittent stimulation (Takaya et al., (1996) Epilepsia 37: 1111-1116), continuous stimulation can cause nerve damage, whereas intermittent stimulation does not (Agnew et al., (1989) Ann. Biomed. Eng. 17: 39-60; Agnew & McCreery, (1990) Epilepsia 31 [Suppl. 2]: S27-S32; Ramsay et al., (1994) First International Vagus Nerve Stimulation Study Group, Epilepsia 35: 627-636). Second, the side effects associated with VNS are typically experienced during the stimulation (Uthman et al., (1993) Neurology 43: 1338-1345; Ramsay et al., (1994) First International Vagus Nerve Stimulation Study Group, Epilepsia 35: 627-636; McLachlan, (1997) J. Clin. Neurophysiol. 14: 358-368), so giving intermittent stimulation reduces their occurrence. However, because stimulation is delivered regardless of whether seizure activity is present or is likely to occur, this fixed stimulation protocol has the disadvantage that the patient may receive excess stimulation and suffer excessive side effects.

The second main problem is that the vagus nerve is involved in, among other things, cardiovascular and abdominal visceral functions. Indeed, because of the pattern of vagus innervation of the heart, the vagus nerve can only safely be stimulated unilaterally (i.e., on the left side only). This is a potential limitation in the efficacy of cranial nerve stimulation because the effects of the stimulation may be bilateral (Chase et al., (1966) Exp. Neurol. 16: 36-49; Zabara (1992) Epilepsia 33: 1005-1012; Henry et al., (1998) Epilepsia 39: 983-990; Henry et al., (1999) Neurology 52: 1166-1173) and may, therefore, be aided by adding more stimulation sites. For these reasons, use of a nerve without the types of visceral fibers that are found in the vagus nerve is more effective for seizure reduction.

Various prior art methods and apparatus purport to reduce or eliminate epileptic seizures. See, e.g., U.S. Pat. No. 6,016,449 to Fischell et al.; U.S. Pat. No. 6,061,593 to Fischell et al.; and U.S. Pat. Nos. 5,540,734; 4,702,254; 4,867,164 and 5,025,807 to Zabara. However, these references do not disclose the stimulation of the trigeminal nerve as an aspect of seizure reduction. Nor do these references disclose an automatic stimulation device that provides stimulation only when a seizure is detected. These and other references appear to generally disclose stimulation of the vagus or other nerves, to the exclusion of the trigeminal nerve. Additionally, these and other references disclose continuous, regular and periodic stimulation of a nerve; they do not disclose stimulation of a nerve exclusively during seizure-related activity. Moreover, these references do not disclose bilateral nerve stimulation which, in the case of vagus nerve stimulation, can be hazardous to a subject's health.

What is needed, therefore, is an apparatus and method of detecting and ameliorating epileptic seizures by stimulation of the trigeminal nerve, either alone or in combination with stimulation of other cranial nerves. Such an apparatus and method preferably automatically triggers stimulation of the trigeminal nerve only when a seizure is present or immediately before the seizure occurs, and that stimulation cease when no seizure is present or imminent. Preferably, the apparatus is adapted to be chronically implanted in a subject. It is also preferable that the apparatus minimize side effects, including cardiac damage. The present invention solves these and other problems and represents a significant advance over prior art methods of detecting and ameliorating seizures.

### SUMMARY OF THE INVENTION

An intelligent brain pacemaker for mammals having a cranial nerve not associated with an autonomic function is disclosed. In a preferred embodiment, the intelligent brain pacemaker comprises (a) one or more electrodes adapted to acquire field potential measurements indicative of a mammal's brain activity in real-time; (b) a seizure detector adapted to detect seizure-related brain activity of a mammal in real-time, the seizure detector being electrically connected to the one or more electrodes; (c) one or more nerve stimulators adapted to provide electrical stimulation to a mammal's cranial nerve not associated with an autonomic function, to terminate or ameliorate the seizure, the one or more nerve simulators being electrically connected to the seizure detector; and (d) a power source for providing power to the intelligent brain pacemaker.

It is preferable that the one or more electrodes comprises one or more microwire arrays comprising polytetrafluoroethylene (TEFLON®)-coated stainless steel or tungsten wires, and it is more preferable that the stainless steel wires are about 50 μm in diameter and that the one or more electrodes comprises an array or bundle of 8 or more TEFLON®-coated stainless steel or tungsten wires. Additionally, it is preferable that the one or more electrodes are adapted to be affixed to exterior of a subject's body, that each electrode is monitored on a separate channel, that field potential measurements indicative of a subject's brain activity are acquired continuously and that seizure-related brain activity of a subject is detected continuously.

It is preferable that the seizure detector is adapted to (a) determine if any field potential measurement matches a known pattern of epileptic brain activity; (b) send a signal to a stimulator if a field potential measurement matches a known pattern of epileptic brain activity; (c) continue sending a signal to the stimulator for as long as the a field potential matches a known pattern of epileptic brain activity; and (d) stop sending a signal to the stimulator when field potential measurements do not match a known pattern of epileptic brain activity and comprises a seizure detection algorithm running on a computer microchip. Preferably, the one or more nerve stimulators comprise a nerve cuff electrode; the electrical stimulation is provided in the form of a pulse train; and one of the branches of the trigeminal nerve is electrically stimulated. It is further preferable that the apparatus is adapted to be implanted in the body or brain tissue of a subject.

A method of detecting and ameliorating a seizure in a mammal having a cranial nerve not associated with an autonomic function is disclosed. In a preferred embodiment, the method comprises (a) acquiring field potential data indicative of a subject's electrical brain activity in real-time; (b) analyzing the field potential data to identify seizure-related brain activity; (c) electrically stimulating a cranial nerve not associated with an autonomic function of the subject, if seizure-related brain activity is identified; and (d) removing the stimulation when seizure-related brain activity is not detected, whereby a seizure is detected and ameliorated. Preferably, the method is performed without generating a detectable cardiovascular side effect and the field potential data is acquired continuously via one or more microelectrode arrays comprising one or more bundles of TEFLON®-coated stainless steel or tungsten microwires, data acquired from each of which is recorded on a separate channel. Preferably, the analyzing comprises the steps of: (a) band-pass filtering the acquired field potential data; (b) comparing the field potential data to a known pattern of epileptic brain activity; and (c) determining if any of the field potential data exceeds the known pattern of epileptic brain activity, wherein the predetermined threshold voltage value is indicative of one of (1) seizure-related brain activity and (2) brain activity predictive of oncoming seizure activity. It is additionally preferable that electrical stimulation is automatically triggered if seizure-related brain activity is identified and is stopped when seizure-related brain activity is absent. Preferably, the one or more nerve stimulators comprise a nerve cuff electrode, the electrical stimulation is provided in the form of a pulse train, that the 10 branch of the trigeminal nerve is electrically stimulated. It is further preferable that the method be performed within the body of a subject.

A method of increasing the time between epileptic seizures is disclosed. In a preferred embodiment, the method comprises: (a) acquiring field potential data from the brain of a subject; (b) analyzing the field potential data to identify the presence of an epileptic seizure in a subject; (c) electrically stimulating a cranial nerve not associated with an autonomic function of the subject when an epileptic seizure is identified; and (d) repeating steps (a) through (c), whereby the time between seizures is increased. Preferably, the method is performed without generating a detectable cardiovascular side effect and the field potential data is acquired continuously via one or more microelectrode arrays comprising one or more bundles of TEFLON®-coated stainless steel or tungsten microwires, data acquired from each of which is recorded on a separate channel. Preferably, the analyzing comprises the steps of: (a) band-pass filtering the acquired field potential data; (b) comparing the field potential data to a known pattern of epileptic brain activity; and (c) determining if any of the field potential data matches the known pattern of epileptic brain activity, wherein the predetermined threshold voltage value is indicative of one of: seizure-related brain activity and brain activity predictive of oncoming seizure activity. It is additionally preferable that electrical stimulation is automatically triggered if seizure-related brain activity is identified and is stopped when seizure-related brain activity is absent. Preferably, the one or more nerve stimulators comprise a nerve cuff electrode, the electrical stimulation is provided in the form of a pulse train, such that a branch of the trigeminal nerve is electrically stimulated. It is further preferable that the method be performed within the body of a subject.

Some of the objects of the invention having been stated hereinabove, other objects will become evident as the description proceeds when taken in connection with the accompanying drawings as best described hereinbelow.

### BRIEF DESCRIPTION OF THE DRAWINGS

FIG. 1 is a schematic diagram of one embodiment of the intelligent brain pacemaker of the present invention.

FIGS. 2A and 2B are an EKG trace of cardiac activity occurring during a period of IO nerve stimulation, indicating that EKG activity is not significantly altered during IO nerve stimulation.

FIG. 2C is a series of EKG traces correlating instantaneous heart rate over a 15 minute period, during which stimulation was twice provided continuously for 1 minutes, as well as five times for shorter bursts.

FIGS. 3A1-3A3 are filtered field potential traces showing seizure activity during three sequential 1 minute periods demonstrating that stimulation of the IO nerve reduces seizure activity in a current-dependent manner. (In FIG. 3A1, representing minute 1, no stimulus is provided; in FIG. 3A2, representing minute 2, stimulus is provided; and in FIG. 3A3, representing minute 3, no stimulus is provided.)

FIGS. 3B-3D are traces depicting average integrated seizure activity, number of seizures and seizure duration, respectively, during 1 minute periods of stimulation at different current levels compared with the period of no stimulation directly preceding each stimulus on period. (In these figures, a solid line connects stimulation-off periods measurements; a dashed line connects stimulation-on values; thick black horizontal lines at 100% denote the level of no change in seizure activity.)

FIG. 4A is a plot depicting the effect of varying the stimulus frequency, using a periodic stimulation paradigm, on the number of seizures observed.

FIG. 4B is a plot depicting the effect of varying the stimulus frequency, using a periodic stimulation paradigm, on the duration of observed seizures.

FIGS. 5A1-5A3 are filtered field potential traces showing seizure activity during three sequential 1 minute periods, demonstrating the effects of bilateral stimulation versus unilateral stimulation. (In FIG. 5A1, representing minute 1, no stimulus is provided; in FIG. 5A2, representing minute 2, stimulus is provided; and in FIG. 5A3, representing minute 3, no stimulus is provided.) FIGS. 5B-5D are traces depicting integrated seizure activity, number of seizures and seizure duration, respectively, during 1 minute periods of bilateral stimulation at different current levels compared with the period of no stimulation directly preceding each stimulus on period. (In these figures, a solid line connects responses contralateral to the stimulation site; a line with long dashes connects responses ipsilateral to the stimulation site; a line with short dashes connects responses to bilateral stimulation.)

FIGS. 6A-6C are traces depicting correlation of seizure detection, nerve stimulation and field potentials for three separate seizure instances, indicating that seizure-specific stimulation stops synchronous activity.

FIGS. 7A1-A3 are filtered field potential traces showing seizure activity during three sequential 1 minute periods, demonstrating seizure reduction using the intelligent brain pacemaker. (In FIG. 7A1, representing minute 1, no stimulus is provided; in FIG. 7A2,representing minute 2, stimulus is provided; and in FIG. 7A3, representing minute 3, no stimulus is provided; the bars on the line labeled “seizure detector” indicate seizures detected by the seizure detection device.)

FIGS. 7B-7D are plots depicting average integrated seizure activity, number of seizures and seizure duration, respectively, over 1 minute periods, during which the seizure detector (i.e. the intelligent brain pacemaker) was activated (which occurred only when a seizure began) at different current levels, as compared with the period of no stimulation directly preceding each stimulus on period. (In these figures, a solid line connects stimulation-off periods measurements; a dashed line connects stimulation-on values; thick black horizontal lines at 100% denote the level of no change in seizure activity.)

FIGS. 8A-8C are plots depicting the amount of seizure reduction versus the amount of stimulation provided. (In these figures, the dashed line indicates stimulation provided by the periodic stimulation paradigm and the solid line indicates stimulation provided by the intelligent brain pacemaker. The Y-axis of the plots represents the ratio of seizure activity reduction to seconds of stimulation in a given stimulation-on period.)

### DETAILED DESCRIPTION OF THE INVENTION

I. Definitions

Following long-standing patent law convention, the terms “a” and “an” mean “one or more” when used in this application, including the claims.

As used herein, the term “band-pass filtering” means a signal processing operation in which unnecessary or undesired frequency signal components, or frequency signal components that might interfere with seizure detection, are attenuated. A band pass filter can be, for example, a notch filter at 30 Hz, 60 Hz, 90 Hz or another desired frequency.

As used herein, the term “bilateral stimulation” and grammatical derivatives thereof means stimulation of two different sites. For example, bilateral stimulation of the trigeminal nerve of a subject having a trigeminal nerve pair can be achieved by stimulating both the right and left branches of the IO branch of the trigeminal nerve of a subject, but not exclusively the right or exclusively the left member.

As used herein, the term “continuous”, and grammatical derivatives thereof, takes its ordinary meaning and means without significant interruption. Something can be “continuous” while still being interrupted by short breaks, however the term excludes the notion of long breaks. Thus, a stream, for example, can be interspersed with short interruptions and still be continuous. In another example, a stream can be interrupted for intervals on the order of microseconds, milliseconds or seconds and can still be considered to be continuous on a given time scale.

As used herein, the term “cranial nerve” means any of the following 12 nerves, each of which is normally present in pairs. The cranial nerves include (the accepted numerical identifier of which is denoted in parentheses) the olfactory nerve (I), the Optic nerve (II), the oculomotor nerve (III), the trochlear nerve (IV), the trigeminal nerve (V), the abducens nerve (VI), the facial nerve (VII), the auditory or vestibulocochlear nerve (VIII), the glossopharyngeal nerve (IX), the vagus nerve (X), the spinal accessory nerve (XI) and the hypoglossal nerve (XII). In the context of the present invention, cranial nerves are preferably disposed in mammals and more preferably disposed in humans.

As used herein, the term “cranial nerve not associate with an autonomic function” means any of the twelve cranial nerves which is not involved or associated with an autonomic function, such as breathing, cardiac rhythms and other functions which occur autonomically. Thus, a vagus nerve, which is associated with cardiac rhythms, is not a “cranial nerve not associated with an autonomic function;” a trigeminal nerve, however, is not associated with an autonomic function and therefore is a “cranial nerve not associated with an autonomic function.”

As used herein, the terms “epileptic seizure” and “seizure” are used interchangeably and mean an involuntary impairment of motor control in a subject. Although seizures are typically coincident with an abnormal hypersynchronization of electrical activity in a subject's brain, the present invention is not limited to identifying the presence of a seizure by detecting hypersynchronous electrical activity in a subject's brain. Indeed, the present invention contemplates employing a variety of seizure indicators that can, but are not required to include, identification of hypersynchronization of brain activity. For example, the present invention discloses that the presence of a seizure can be identified by a gradual or a sudden increase in the amplitude of electrical brain activity; such an increase can be due exclusively to hypersynchronization of electrical activity, but can additionally be due to other factors exclusively, or in combination. On a macroscopic scale, a variety of physiological effects can indicate the presence of a seizure in an individual, and can range from twitching and fidgeting to a stiffening and shaking of the limbs and loss of consciousness.

As used herein, the terms “field potential data” and “field potentials” are used interchangeably and mean voltage measurements collected from one or more locations in a subject's brain or nervous system.

As used herein, the term “known pattern of epileptic brain activity” means a pattern of brain activity known to be associated with an epileptic condition. The term can refer to brain activity occurring before or during a seizure that is recognized as activity associated with an epileptic condition. The term specifically encompasses field potential values that exceed a predetermined threshold value. A “known pattern of epileptic brain activity” can be stored on a data storage medium and can serve as a standard against which brain activity data can be acquired from a subject and compared to determine if epileptic brain activity is present in the subject. Summarily, a “known pattern of epileptic brain activity” means brain activity that is known to occur before or during an epileptic seizure.

As used herein, the term “microwire array” means collection of two or more microwires, the microwires having a first and a second end. The first end of a microwire is preferably, but not required to be, adapted to interact with neural tissue and the second end is preferably disposed in electrical communication with a head assembly, the head assembly adapted to coalesce signals acquired by each microwire of a microwire array. Preferably the second end of the each microwire is maintained in a fixed spatial relationship with other microwires of the microwire array.

As used herein, the term “nerve contact electrode” means an electrode that is in direct contact with a nerve to be stimulated. A nerve cuff electrode is one embodiment of a nerve contact electrode.

As used herein, the term “nerve cuff electrode” means an electrode adapted to circumferentially encircle a nerve and deliver an electrical stimulus to the nerve it encircles.

As used herein, the term “nerve stimulator” means any device or means adapted to stimulate one or more nerves. Stimulation imparted by a nerve stimulator can be of an electrical, optical or physical nature, however electrical stimulation is preferred.

As used herein, the terms “operator”, “patient” and “subject” are used interchangeably and mean any individual monitoring or employing the present invention, or an element thereof. Operators can be, for example, researchers gathering data from an individual, an individual who determines the parameters of operation of the present invention or the individual in or on which the intelligent brain pacemaker is disposed. Broadly, then, an “operator”, “patient” or “subject” is one who is employing the present invention for any purpose. As used herein, the terms “operator”, “patient” and “subject” need not refer exclusively to human beings, but rather the terms encompass all organisms having a cranial nerve not associated with an autonomic function, such as organisms having a trigeminal nerve.

As used herein, the term “pulse train” means a series of pulses. For example, a pulse train can comprise electrical pulses interrupted at regular or irregular intervals by an absence of electrical energy.

As used herein, the term “seizure detection algorithm” means an operation comprising one or more steps which, when performed and the results are analyzed, can indicate whether a seizure is occurring, is not occurring, is predicted to occur or is not predicted to occur. A seizure detection algorithm can comprise, for example, the steps of comparing brain activity of a subject to a database of brain activity known to be associated with seizure activity. A seizure detection algorithm can also comprise, for example, the steps of comparing brain activity of a subject to a threshold voltage value, above which seizure activity is known to be occurring.

As used herein, the term “seizure-related brain activity” means brain activity, for example electrical activity within the brain, known or suspected to be indicative of the presence or onset of a seizure.

As used herein, the term “threshold voltage value” means a minimum or maximum voltage. A threshold voltage value can be expressed in volts, millivolts, microvolts or other convenient units.

As used herein, the term “trigeminal nerve” means the fifth pair of cranial nerves. Physiologically, the trigeminal nerve is generally implicated in chewing and in face and mouth pain and touch.

As used herein, the term “unilateral stimulation” and grammatical derivatives thereof means stimulation of a given nerve on only one side of a subject's head or body. For example, unilateral stimulation of the trigeminal nerve of a subject having a trigeminal nerve pair can be achieved by stimulating either the right or left nerve (or subbranches thereof, such as the IO branch) of the subject, but not both branches.

II. General Considerations

The trigeminal nerve is the largest of the cranial nerves and comprises three main branches, the ophthalmic branch, the maxillary branch and the mandibular branch. The ophthalmic branch, also known as the VI sensory branch, comprises a series of subbranches including the infratrochlear branch, the anterior ethmoid branch, the posterior ethmoid branch, the lacrimal branch, the supraorbital branch, the supratrochlear branch and the nasociliary branch.

The maxillary branch, also known as the V₂ sensory branch, comprises a series of subbranches including the zygoticaticotemporal branch, the zygomaticofacial branch, the posterior superior alveolar branches, the nasopalatine branch, the greater and less palatine branches, the mid and anterior alveolar branches and, of significant interest in the context of the present invention, the infraorbital, or simply “IO”, branch.

The mandibular nerve (also known as the V₃ sensory motor branch) branches include the auriculotemporal branch, the lingual branch, the inferior alveolar branch, the mental branch and the buccal branch. Although one embodiment of the present invention recites stimulation of the infraorbital branch of the trigeminal nerve specifically, the present invention contemplates stimulation of any of the recited sub branches of the three large branches of the trigeminal nerve. The trigeminal nerve is a cranial nerve not associated with an autonomic function, such as cardiac rhythms, breathing, etc. Thus, unlike a cranial nerve associated with an autonomic function (e.g., the vagus nerve), the trigeminal nerve can be stimulated unilaterally or bilaterally without adverse effects on an autonomic function.

In one aspect of the present invention, field potential measurements are acquired. Field potential measurements are measurements of the electric field potential of an area or region of the brain or other organ. The field potential detected at any one point represents the sum of the potential created by an number of electric potential generators in the area surrounding the field potential measuring device.

By way of example, when an individual monitors a field potential (i.e. the amplitude of a field potential) at a point on the surface of the cerebral cortex, for example, what is detected is the overlapping summation of electric fields generated by active neurons in the depths of the cerebral cortex, which have spread through the tissues and up to the surface. These nerve cells can be characterized as point dipoles that are oriented perpendicular to the surface of the cerebral cortex. In other words, each cell has a current source where positive charge moves outwardly across its membrane and a current sink where the same amount of positive charge moves inwardly at each instant. Thus, the flow of current across each cell establishes an electric field potential that is equivalent to the electrostatic field potential of a pair of point charges, one positive at the location of the current source and one negative at the current sink. The amplitude of this field potential, i.e., the electric field strength, decreases inversely with distance in all directions from each point charge, and is relatively low at the surface of the cerebral cortex.

When many nerve cells are generating field potentials in a given region, these field potentials sum and overlap in the neural tissue, in the extracellular fluid, and at the brain surface. This summation is a linear function in this volume conductor, since the field strength of a given cell varies inversely as a function of the distance from each current source or sink. Thus, if the electric potential of a given region is measured at a sufficient number of points and depths, it is possible to deduce the locations and amplitude of each dipole generator at any instant of time.

III. Configuration and Operation of the Intelligent Brain Pacemaker

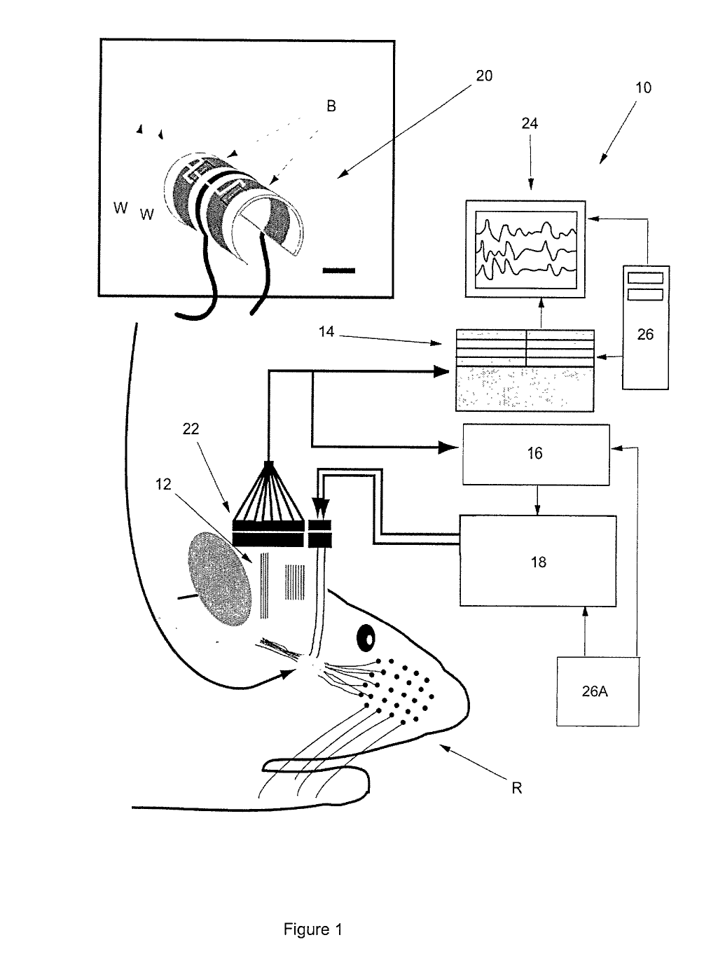

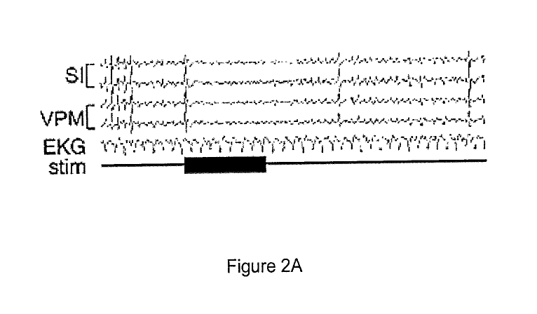

FIG. 1 is a schematic drawing of one embodiment of an intelligent brain pacemaker of the present invention, generally designated 10, depicting a configuration of the seizure detector, nerve stimulator, placement of the nerve cuff electrode and positioning of the microwire array. Specifically, in this embodiment, intelligent brain pacemaker 10 is disposed in rat R, as indicated. However the intelligent brain pacemaker can also operate in other mammals having a trigeminal nerve, such as human patients. Broadly, when the device depicted in FIG. 1 is in operation, field potential signals from chronically implanted microwires 12 in the subject's brain are sent to an amplifier and recording unit 14 for collection, as well as to ASD (automatic seizure detector) 16. When ASD 16 detects seizure activity, it sends a signal to stimulator 18, which delivers a current pulse to implanted nerve cuff electrode 20 which, in turn, stimulates the nerve in contact with nerve cuff electrode 20. Lead wires W and platinum bands B are denoted in FIG. 1.

Referring again to the embodiment depicted in FIG. 1, a more detailed description of the how the intelligent brain pacemaker is configured and operates is as follows. Initially, microwire array 12 is implanted in the brain of the subject mammal. In the embodiment of FIG. 1, the microwire electrodes converge at head stage 22. Each electrode is afforded its own channel. The field potential data sensed by the electrodes is then transmitted to two different locations: automatic seizure detector 16 and to signal amplifier 14. The field potential data transmitted to signal amplifier 14 is then recorded, amplified and displayed on computer screen 24 for visual inspection. In this embodiment, the signal amplifier 14 is an integrated component of computer 24 on which the data is stored, processed and visualized. In this embodiment, there is no interaction between signal amplifier 14 and associated computer 24 and seizure detector 16. These data paths are independent of one another in the configuration of FIG. 1. An advantage of displaying the data on computer screen 24, which is displayed in real time, is that it affords an observer an opportunity to view the data as it is received.

The field potential data are concurrently transmitted to automatic seizure detector 16. Automatic seizure detector 16 then band-pass filters the field potential data incoming from each channel (i.e. electrode) of microwire array 12 and compares the resultant field potential values to a threshold value. Field potentials exceeding the threshold value are interpreted to be indicative of the presence of a seizure.

Continuing with the embodiment depicted in FIG. 1, when a field potential value surpasses a threshold value or is indicative of a known pattern of epileptic brain activity, indicating the presence or onset of a seizure, a signal, such as a TTL pulse, is automatically sent to nerve stimulator 18. Nerve stimulator 18 then delivers an electrical pulse, or a series of electrical pulses, to nerve cuff electrode 20. The nature and character of the electrical pulse or pulses delivered by nerve stimulator 18 can be varied and the nerve stimulator programmed accordingly.

The electrical pulse or pulses generated by nerve stimulator 18 then follow lead wires W to the nerve contact electrode. In FIG. 1, the nerve contact electrode is nerve cuff electrode 20. The electrical pulses are transmitted through conductive medium B to the nerve that is to be stimulated. FIG. 1 depicts the IO branch of a trigeminal nerve being stimulated.

The electrical pulses delivered to the stimulated trigeminal nerve serve to disrupt hyper-synchronous activity and thereby ameliorate an oncoming or occurring seizure. The electrical pulse or pulses can be applied as long as the seizure is present. Stated another way, electrical pulses can be applied as long as incoming field potential measurements surpass the threshold voltage value.

A power source 26 is provided to power computer 24 and signal amplifier 14, an optional component of intelligent brain pacemaker 10. Power source 26 can be disposed outside the body of a patient employing the intelligent brain pacemaker or can be implanted subcutaneously. Power source 26A is provided to power the nerve stimulator 18 and ASD 16. Power source 26A is preferably adapted to be implanted in or on the body of a patient employing intelligent brain pacemaker 10. Although many different types of power sources 26 and 26A can be employed in the invention, preferred power sources include lithium batteries.

The configuration disclosed in FIG. 1 for rat R can be modified to accommodate a human subject. Additional modifications, such as the omission of computer 24 and signal amplifier components 14, can be made and suitable modifications will be apparent to those of skill in the art upon consideration of the present disclosure.

III.A. Automatic Seizure Detection and Amelioration

An advantage of the intelligent brain pacemaker is that it automatically detects and reduces seizures, without the need for human intervention. That is, once in place, the intelligent brain pacemaker can function autonomously to detect and reduce seizures with no need for input from a human, including the individual in or on whose body the intelligent brain pacemaker is situated. Thus, an advantage of the present invention is its ability to function autonomously and without human intervention.

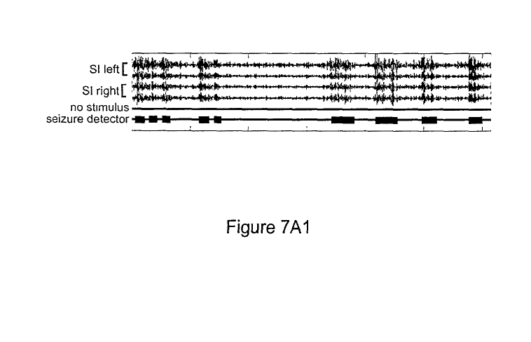

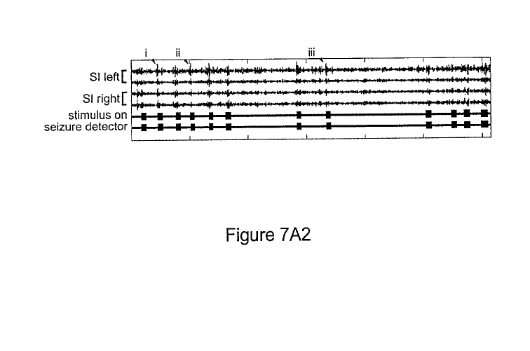

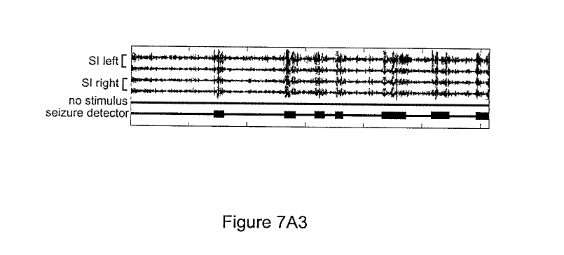

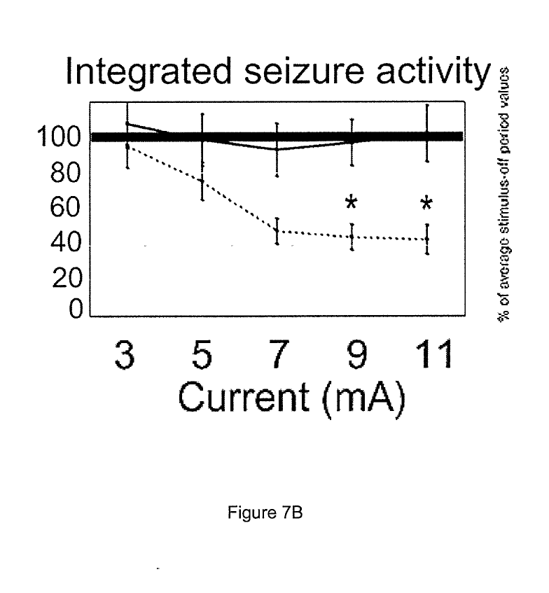

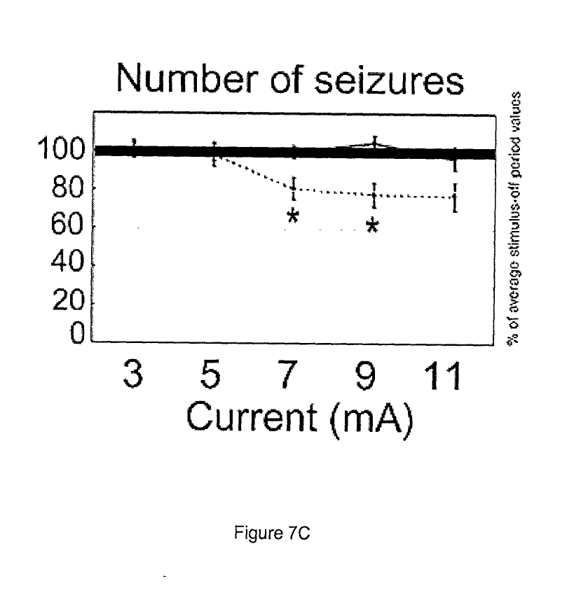

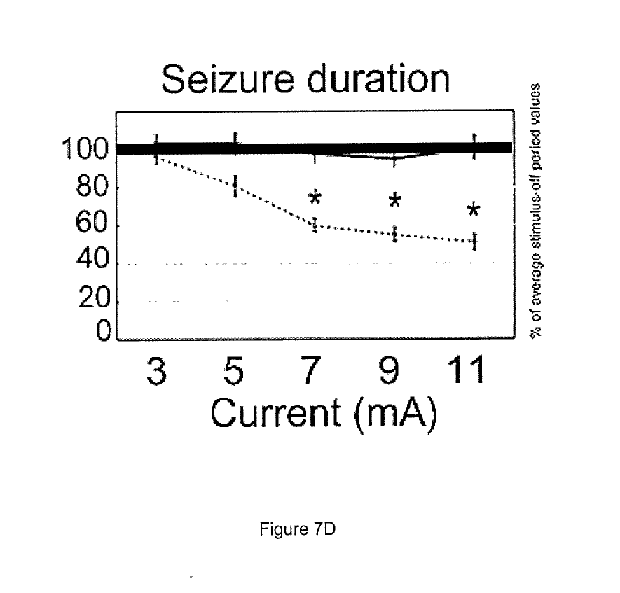

FIGS. 7A-7D demonstrate the automatic seizure detection and reduction aspect of the present invention. FIGS. 7A-7D demonstrate seizure reduction by the intelligent brain pacemaker. FIGS. 7A1-7A3 depict filtered field potential traces showing seizure activity during three sequential one minute periods. FIG. 7A1 depicts no stimulus; FIG. 7A2 depicts stimulus on; and FIG. 7A3 depicts no stimulus. The stimulus parameters giving rise to the data displayed in these figures were 9 mA, 333 Hz and a 0.5 msec pulse duration. Within each segment, the trace labeled “seizure detector” indicates where the seizure detector detected seizure activity. The trace labeled “stimulus on” indicates where the seizure detector sent a TTL pulse to trigger the IO nerve stimulator when it detected such activity. Thick black horizontal lines at 100% denote the level of no change in seizure activity. Calibration for these figures is: 200 μV, vertical and 10 sec, horizontal.

FIG. 7B represents the effect of stimulation on integrated seizure activity, FIG. 7C represents the effect of stimulation on the number of seizures observed and FIG. 7D represents the seizure duration. Error bars represent +/− SEM. Solid lines connect stimulation-off values; a dashed line connects stimulation-on values. Stimulation-on values significantly different from stimulation off values are designated by an asterisk in the figures. Thick black horizontal lines at 100% denote the level of no change in seizure activity. Calibration for these figures is 200 μV, vertical and 10 sec, horizontal.

The data presented in FIGS. 7A-7D was acquired without human intervention. In FIGS. 7B-7D, the dotted lines represent data acquired without human intervention. That is, the seizure detector automatically detected the presence of a seizure and automatically sent a TTL pulse to the nerve stimulator which applied an electrical pulse to the nerve in contact with the nerve contact electrode. Note also that upon the subsidence of a seizure, the seizure detector stopped sending signals to the nerve stimulator. Additionally, the seizure detector sent no further TTL pulses until another seizure was detected, at which point another TTL pulse was sent to the nerve stimulator. This entire operation was performed automatically, with human interaction entering only when the acquired data was analyzed. Therefore, an advantage of the present invention over prior art seizure detectors is the ability of the intelligent brain pacemaker to work automatically and independently of human interaction, similar to cardiac pacemakers known in the art.

IV. Embodiments and Optional Components

The above sections disclose the core elements and operation of the intelligent brain pacemaker. However, configurations employing additional components and embodiments are possible; suitable components and embodiments will be apparent to those of skill in the art upon consideration of the present disclosure. For example, a chart recorder (not shown) can be attached to a signal amplifier to generate a real time paper record of brain activity detected by electrodes acquiring field potential data.

In addition, additional seizure detection algorithms can be employed by an ASD of the present invention. Such algorithms can be adapted to operate independent of the signal voltage for the identification of a seizure, for example, and can also (or instead) rely on other parameters for seizure detection, such as signal frequency components.

IV.A. Signal Amplifier

It can be desirable to employ a signal amplifier to record and display the amplitude of field potential data on a computer screen, although there is no requirement that a signal amplifier form a component of the intelligent brain pacemaker. The visualization of field potential data can be accomplished, in part, by employing a signal amplifier. A signal amplifier can magnify the amplitude of the raw field potential data and can facilitate storage of this data either onboard the signal processor or on an associated computer.

A signal amplifier adapted for use in the present invention preferably has one channel for each electrode, and data for each electrode is recorded and treated by the signal amplifier separate from the data acquired from other electrodes. Thus, a signal amplifier can comprise 16 channels, one for each microwire electrode. As noted, field potential data acquired from a subject is preferably stored by the signal amplifier in a channel-by-channel format, thereby enabling an operator to identify the tissue source of the signal.

A signal amplifier amplifies and preferably records field potential data acquired by the electrodes. In one mode of operation, field potential data, i.e. voltage measurements, is acquired by the conductive electrodes and is then transmitted to a signal amplifier, optionally through a head stage first. The field potential data, which is electrical data typically in the form of voltage amplitudes, can then be recorded channel-by-channel and stored on a suitable device, such as a personal computer.

A signal amplifier can also serve to enhance the field potential data to a level at which it can be analyzed and treated by operators. Preferably, the signal amplifier introduces a minimum of noise to the signal. Amplification of the field potential data can occur prior to or after the signal is recorded by the amplifier.

Although the electrodes can continually detect field potential data, a signal amplifier can intermittently record data. That is, measurements of field potential data can be stored and amplified at regular intervals. The interval, or rate, at which field potential data is acquired is known as the sampling rate. Suitable sampling rates will be apparent to those of skill in the art, upon contemplation of the present disclosure, although a sampling rate of 512 Hz is preferred. The sampling rate can be set on the signal amplifier. Thus, the signal amplifier can record and amplify data at the sampling rate set by an operator. Suitable amplifiers include the GRASS Model 15 amplifier (available from Grass Instrument Co. of Quincy, Mass.).

Field potential data can also be visualized on a computer screen. This ability can be of assistance to operators wishing to identify the character of field potential data giving rise to seizures and seizure-related activity. To facilitate this analysis, a signal amplifier can be disposed onboard a computer itself or, alternatively, a signal amplifier can be a portable standalone unit capable of recording and processing field potential data at any desired time. The latter mode offers the advantage of freeing a subject from the need to be continuously in the vicinity of a computer in order to visualize field potential data. Such a unit is preferably adapted to download stored data to a computer at the convenience of an operator. This aspect of the present invention can be useful when the intelligent brain pacemaker is an integrated device, with no requirement that a signal amplifier interact with a computer, beyond transient data storage. It is important to note, however, that there is no requirement that the intelligent brain pacemaker comprise a signal amplifier in order to operate.

IV.B. Roles for a Computer in the Present Invention

A computer can form an optional component of the present invention. The computer can function as an aid in the visualization of field potential data, which can then be displayed on a monitor associated with the computer. Additionally, a computer can be employed to perform any desired analytical processes on acquired field potential data. For example, a computer can be employed to perform a subsequent analysis of the effectiveness of a given threshold value or to perform a quantitative or qualitative analysis of acquired field potential data. A computer can be deployed in addition to the seizure detector, as depicted in FIG. 1, and can essentially run in parallel to the seizure detector. In another configuration, which might be suitable in a laboratory setting, the seizure detector and/or the nerve stimulator can form a component of the computer, thereby enabling detection and analysis on a single unit.

IV.B.1. Intranet and Internet Capability

A computer forming a component of the intelligent brain pacemaker can be fitted with internet and/or intranet capability. This embodiment of the present invention can facilitate direct transfer of field potential data and other parameters to another computer situated locally or remotely. For example, if a computer is fitted with intranet capability, this will facilitate the transmission of data from the local computer to another computer on the intranet, such as a computer disposed in another laboratory or other location within the building in which a subject is situated. Alternatively, the computer can be fitted with internet capability, thereby permitting transmission of data to a computer at a remote location. In this embodiment, signals from a subject's brain, which can comprise data related to the nature and quality of a subject's seizure attack, can be sent via a network (such as an internet) to the subject's physician, who can then evaluate the severity and nature of the seizure. Other uses for a computer in the context of the present invention will be apparent to those of skill in the art upon consideration of the present disclosure.

IV.B.2. Handheld Computers

Computers useful for practicing the present invention need not be personal computers, although personal computers might be preferable for monitoring a subject in a laboratory setting. Handheld computers might be more practical when the present invention is disposed in a patient who is employing the intelligent brain pacemaker in day-to-day life. The intelligent brain pacemaker does not require significant computing resources and, thus, a handheld computer might be sufficient to meet the needs of a patient in the same way a personal computer might.

Additionally, a handheld computer might be useful for a supervising physician or care provider to monitor an operator's epileptic activity. In practice, it might be desirable to examine field potential data and epileptic activity from an operator. To meet this desire, the data stored on the handheld computer could be downloaded to another computer for analysis. For example, an operator might schedule regular visits with his or her physician to determine the efficacy of the operating parameters (e.g., threshold voltage value, pulse train parameters, etc.) of the present invention. During these visits, the physician might download data stored on the handheld computer for later analysis and an assessment of the operating parameters of the present invention. Analysis of the data might indicate that the threshold voltage value should be set higher or lower for maximum effectiveness. The small size of a handheld computer makes it easy to interface with the intelligent brain pacemaker and to carry on the operator's person.

IV.B.3. Radio Telemetry Transmission of Field Potential Data and Seizure Activity

When practicing the present invention, it might be desirable to wirelessly transmit data from the seizure detector or nerve stimulator to a computer, which, again, might be a personal computer, or a handheld computer. Radio telemetry circuitry, therefore, can comprise an element of the present invention. In this embodiment of the present invention, radio telemetry circuitry can be disposed in or on an operator's person. This circuitry might function to transmit field potential or other data from the seizure detector or signal amplifier to a data storage unit, such as a handheld computer, for later downloading and/or analysis. Similarly, radio telemetry circuitry might be employed to transmit signals to the seizure detector or signal amplifier in order to fine-tune various operational parameters, such as threshold voltage value or pulse train characteristics.

IV.B.4. Suitable Software Packages

A variety of software packages can be employed in the operation of the intelligent brain pacemaker. Suitable software packages can be written de novo or can be purchased commercially and modified to fit the needs of an operator. When data analysis software is custom-made it can be developed using a variety of platforms, such as MATLAB®, which is available from The Mathworks, Inc. of Natick, Mass. Software packages that can be useful for practicing the present invention can include software for data acquisition and data analysis. Such software can reside on the seizure detector or signal amplifier or can reside on a computer employed in a role such as those disclosed hereinabove.

Data analysis software can assist in the visualization of data, interpretation of data and can assist in performing associated statistical analyses, if such analyses are desired. Data analysis software can also aid in an assessment of the efficacy of the operating parameters of the intelligent brain pacemaker. Furthermore, data analysis software can be employed as a mechanism of troubleshooting the present invention when it is disposed in the body of a subject. This can be especially helpful when a physician is contemplating additional surgery to correct a problem with an implanted intelligent brain pacemaker; often data analysis software can function in a diagnostic role. Many times, when a problem is sufficiently identified, it is possible to correct without the need for additional surgery. For example, an unsatisfactorily low threshold voltage value can be identified using data analysis software and possibly corrected without surgery.

Data acquisition software can be of use in setting and altering various operational parameters such as the threshold value, monitoring or changing band-pass filter values or altering the character of a stimulatory pulse train. Such software can be run on a personal computer or on a handheld computer, as circumstances dictate.

IV.B.5. Computer-Intelligent Brain Pacemaker Interfaces

In yet another embodiment of the present invention, an interface can be employed to enable the downloading of data from the seizure detector or signal amplifier directly to a computer. Such an interface can comprise a structure disposed on the body of a subject. Such a structure might also be adapted to interface by cable or radio telemetry with a computer.

IV.C. A Self-Contained, Autonomous Intelligent Brain Pacemaker

In a preferred embodiment of the intelligent brain pacemaker, the invention is entirely disposed in or on the body of an operator. This embodiment of the present invention frees the operator from any need to remain within the vicinity of a given piece of external equipment, for example an external seizure detector or nerve stimulator. In this embodiment, the seizure detector, the signal amplifier and the nerve stimulator are all self-contained and reside in or on the person of the operator. For example, microwire electrodes can be implanted in the cortex or other region of an operator's brain. The electrodes can then interface with the seizure detector. These components can be fashioned with very small dimensions, making them suitable for implantation in the body of the operator. Similarly, the nerve stimulator, which interfaces with the seizure detector, can also be disposed in the body of the subject and can be fashioned of very small dimensions. The implanted pacemaker would be powered by a suitable self-contained power source such as a lithium battery. In this embodiment of the intelligent brain pacemaker, it is important to consider the physiological effects of the implantation of the components of the present invention. Concerns such as biocompatibility issues will be apparent to those of skill in the art and can be addressed accordingly.

V. Real-Time Acquisition of Field Potentials From the Brain of a Subject

In one aspect of the intelligent brain pacemaker, real-time measurements of field potentials are acquired from various points in the brain or neural tissue of a subject. The acquisition of real-time field potential measurements permits the real-time evaluation and analysis of field potential data. Thus, real-time data acquisition and analysis enables an ongoing evaluation of data in the same time frame as the data is acquired. When real-time data acquisition and analysis is performed, there is no delay between data acquisition and the ability to access, analyze and evaluate the acquired data.

As disclosed hereinbelow, the present invention makes possible a variety of real-time field potential data acquisition methods. For example, the present invention discloses the use of microwire electrode arrays and microwire electrode bundles to acquire field potential data in real time. Microwire arrays and bundles are preferred for the acquisition of field potential data. However, any suitable electrode can be employed, such as electrodes disposed on the surface of a patient's skin.

In the context of the present invention, field potentials, which are electric signals, are conducted by electrodes through the electrodes to a terminus where the signals are analyzed. Therefore, suitable electrodes for practicing the present invention will be conductive and, if the electrodes are to be implanted in the tissue of subject, biocompatible with a body and tissues of the subject. As disclosed hereinbelow, however, electrodes need not be implanted and can be secured on the skin of a subject. These electrodes will also comprise a conductive material. Stainless steel wires and tungsten wires are particularly preferred electrodes.

V.A. Preparation of Microwire Electrodes

Microwire electrodes can be employed in the present invention to detect electrical activity, such as field potential measurements, in the brain or neural tissue of a subject. In a preferred embodiment a microwire array comprises a plurality of stainless steel or tungsten microwires. Preferably, the microwires have a diameter of about 50 μm, making them suitable for implantation with a minimum of tissue disruption. Suitable microwires can be manufactured using standard wire pulling techniques or can be purchased commercially from a vendor, such as NBLabs of Denison, Tex. Suitable microwires electrodes can be formed of a conductive material, such as stainless steel or tungsten.

When the microwire electrodes are to be implanted in the brain tissue of a subject, it is preferable to coat the exterior of the microwire electrodes with polytetrafluoroethylene (marketed by DuPont, Inc. of Wilmington, Del. under the trade name TEFLON®) or other insulating material. TEFLON® coating of the microwire electrodes offers a degree of insulation for the microwires, which not only isolates the surrounding tissue from the microwire material but also permits a more spatially-focused determination of field potential. Coating the microwires offers the additional advantage that field potential data can be acquired exclusively from that area of the microwire which is not coated (i.e. the non-insulated cross-sectional area at the end of the implanted end of the microwire electrode).

V.B. Microwire Electrode Arrays

Microwire arrays useful for acquiring field potential data in the context of the present invention can be formed generally as follows. Initially, a plurality of suitable microwires, such as those disclosed herein, are provided. Microwire electrodes have first and second ends: the first end is defined as the end of the electrode that, when emplaced, contacts the brain or neural tissue, while the second end of the electrode ends at a terminus such as an interface with a head stage, signal amplifier or other equipment.

Preferably, at least 8, and more preferably 16 or more, microwire electrodes form a microwire array. It is also preferable, but not required, that the second end of each microwire electrode be fixed in a definite spatial relation to the second ends of other microwire electrodes. This arrangement can be conveniently maintained by employing a head stage into/onto which each second end is affixed. Although the second end of a microwire electrode can be fixed in a head stage, signal amplifier or other piece of equipment, the first end preferably remains flexible, thus facilitating placement at a range of locations.

Thus, a microwire electrode array preferably comprises a plurality of microwire electrodes having free and flexible first ends, while having second ends oriented in a particular spatial arrangement. An advantage of the orientation of the second ends of the microwire electrode is that field potentials can be recorded in a channel-specific fashion, due to the ability to easily correlate the position of a microwire electrode in situ with the position of the second end of the microwire electrode in the terminus.

It is preferable that each microwire electrode be monitored on its own channel, so as to avoid a global average of field potentials for all of the microwire electrodes. By monitoring each electrode on its own channel, it is possible to simultaneously monitor a variety of regions of tissue in a single subject's brain and thus more efficiently monitor a subject for the presence of seizure-related activity.

V.C. Microwire Electrode Bundles

The present invention can also be practiced using microwire electrodes arranged as a bundle, as an alternative to the microwire electrode array disclosed herein, for acquiring field potential data. When a microwire electrode bundle is employed, the microwires preferably are manufactured of a conduction material, such as stainless steel or tungsten, and are at least partially TEFLON® coated.

The microwire electrodes of a bundle will also have first and second ends. The first end or each electrode member of the bundle contacts the tissue, while the second end interfaces with a head stage or signal amplifier. However, unlike the electrodes of an array, the individual electrodes of a microwire electrode bundle are secured in a bunch and it is presumed that all members of the bundle will be implanted in the same general location in a subject's brain tissue, the bundle being considered a single unit for implantation purposes.

A microwire electrode bundle comprises a plurality of microwire electrodes. Each individual member of the bundle can, but need not be, be of a different length. When a microwire bundle comprising electrodes of different lengths is implanted in brain or other tissue, each electrode of the bundle is generally localized to a single region of tissue, however the different lengths of each microwire electrode facilitates acquisition of field potential data at a different tissue depth, effectively providing a depth profile of field potential measurements. Comparing microwire arrays and bundles, the arrays permit data acquisition from multiple sites, while the bundles typically permit data acquisition from multiple depths of the same site.

Like the microwire electrode array, it is preferable that each microwire electrode of a microwire bundle be monitored as a separate channel. This practice facilitates the monitoring of brain tissue at different depths on an electrode-by-electrode basis, as opposed to monitoring the brain tissue as a global average of field potential units.

V.D. Microwire Electrode Array and Bundle Head Stage

The second end of each microwire electrode, which is not the end interacting with tissue (the first end) is preferably affixed to a terminus, such as a conductive port or a conductive material on the head stage. This is preferable regardless of whether the electrodes are arranged in an array or a bundle. Suitable head stages are available commercially (e.g., from NBLabs of Denison, Tex.; head stages pre-equipped with microwire electrodes are also available from this source) or can be manufactured in-house.

A preferable head stage facilitates the communicative attachment of microwire electrodes to the head stage such that field potential data for each electrode is recorded on a separate channel. Preferably, the head stage also has ports or contacts for ground wires. Additional circuitry requirements, such as the need for any resistors or other components should also be considered when selecting or manufacturing a head stage for use in the present invention.

The various ports or contact points on a head stage can be arranged in any desired fashion. For example, when a microwire array is employed, the ports for the microwire electrodes can form an array, for example a two-by-eight array, a one-by-eight array, a four-by-four array or a one-by-sixteen array. By labeling or noting which port is associated with each individual microwire electrode, it is possible to correlate the position of each electrode in the tissue of a subject with the channel on which data for that electrode is being acquired. A head stage can also function as a “collector” of signals and can assist in the orderly transmission of the signals to downstream components of the intelligent brain pacemaker.

V.E. Less-Invasive and Non-Invasive Data Acquisition Apparatuses

The above discussion has focused primarily on the use of microwire arrays and microwire bundles, each of which is preferably implanted directly in the brain or other nervous tissue of a subject, the present invention is not limited to these methods of acquiring field potential data. However, less-invasive and non-invasive methods and apparatuses can also be employed in the present invention in order to acquire field potential data.

A representative non-invasive apparatus for the acquisition of field potential data involves placing suitable electrodes on the scalp or other exterior position of a subject's skin proximate to the organ, structure or region from which field potential data is to be acquired. Suitable electrodes can be fixed in place, for example, by employing a temporary adhesive. However, it is important that once placed an electrode is not free to move, since movement might decrease the quality of field potential data acquired from the electrode. Suitable electrodes can be purchased commercially.

Alternatively, a less invasive approach can be taken with respect to electrode positioning and emplacement. For example, in lieu of placing electrodes directly in the tissue of a subject's brain, electrodes, including microwire electrodes, can be placed subdurally, thereby circumventing the need to insert electrodes into the brain itself. When electrodes are placed subdurally, it is preferable that the electrodes be positioned in areas known or suspected of being implicated in epileptic seizures, as described more completely herein. When placing electrodes subdurally, at least one craniotomy will still be performed, although in this method there is no requirement that the electrodes be placed directly in contact with cortex or other brain or neural tissue.

A less invasive alternative to placing electrodes for acquiring field potential data directly in contact with brain or neural tissue is the use of sub-skin emplacement of electrodes. In this approach, electrodes are implanted under the skin of a subject, for example under the scalp of a subject, in the proximity of regions of the subject's brain or other neural tissue known or suspected to be implicated in seizure-related activity. This approach obviates the need for performing a craniotomy. The small dimensions of the microwires also make this form of emplacement an attractive option.

A variety of types of electrodes can be employed in the disclosed less-invasive and non-invasive methods. For example, microwire electrodes can be employed in the subdural and sub-skin approaches. Microwire electrodes can also be employed in non-invasive approaches as well. However, the more spatially distant an electrode is located from the region it is to monitor, the more sensitive the electrode needs to be. Restating, in non-invasive approaches it is preferable to employ a more sensitive electrode than those electrodes that are to be placed directly in contact with tissue. Preferred electrodes for use in non-invasive approaches can be electrodes of larger dimensions than a microwire electrode, or of greater sensitivity. Additionally, signal amplifiers can help to compensate for any observed low signal amplitudes.

V.F. Surgical Implantation and Surface Attachment Techniques

The electrodes of the intelligent brain pacemaker can be implanted directly in the brain tissue of a subject. The exact positioning (i.e. location and depth) of each electrode can critical and can be determined based on known coordinates. For example, suitable coordinates for placement of electrodes in a rat brain are disclosed in Paxinos & Watson, (1986) The Rat Brain, Ed. 2. New York, Academic, Harcourt, Brace and Jovanovich. When microwires are employed as electrodes, implantations can be made by performing craniotomies in the areas in which electrodes are to be implanted. Craniotomies and implantations can be performed using standard surgical techniques. See, e.g., Nicolelis et al., (1997) Neuron 18: 529-537, incorporated herein by reference.

When microwires are implanted to serve as electrodes, the microwires can be implanted in any region of a subject's brain or other neural tissue. When electrodes are to be implanted in the brain tissue, however, it is preferable that the electrodes be implanted in the primary somatosensory cortices (SI) and/or other regions of the subject's brain. When the primary somatosensory cortices are selected as an electrode placement site, it is preferable that the electrodes be implanted in layer V of the cortices. Additionally, it is preferable that the electrodes be implanted contralaterally, ipsilaterally or both contralaterally and ipsilaterally to the nerve to be stimulated (i.e. the trigeminal nerve).

Employing the following steps, which can be varied at the discretion of the individual implanting the electrodes, electrodes can be implanted in the brain or neural tissue of a subject. Initially, the subject is anesthetized. Craniotomies can then be made in the skull of the subject and the electrodes lowered into the tissue. Various readings can be acquired during the implantation procedure to ensure that the electrodes are placed at the proper depth in the tissue. The precise positioning for each craniotomy can be determined by evaluating coordinate maps, prior to insertion of the electrodes. When the electrodes are properly emplaced, they can be held in place by skull screws, a suitable cement or combinations thereof. Polyethylene glycol, or another biocompatible material, can be employed to coat a microwire before it is inserted into a subject's neural tissue. This practice can assist in the insertion process and is not harmful to the subject, since the material itself is eventually removed from the inserted microwire by mechanisms of the subject's body.

VI. Automatic Seizure Detector

Another component of the present invention is an automatic seizure detection device (“seizure detector” or ASD). Broadly, this component of the intelligent brain pacemaker performs a real time analysis of incoming field potential data and determines if a seizure is occurring or is predicted to occur. When the seizure detector determines that a seizure is occurring or is predicted to occur, it sends a signal to a nerve stimulator to disrupt or counteract the seizure. The automatic seizure detector operates in real time and does not require manual triggering of a signal or any intervention by a human.

VI.A. The Modular Component Parts of the Seizure Detector

An automatic seizure detector preferably comprises the following general modular components: a band-pass filter module, a seizure detection module and a transistor-transistor logic (TTL), or more simply a “digital”, pulse generating module. Broadly, an automatic seizure detector and its modular components can comprise a single integrated unit on a computer microchip or circuit board. The automatic seizure detector can exist as a standalone unit or can be integrated with the electrodes, optional head stage, stimulator or other components of the intelligent brain pacemaker.

The components of an automatic seizure detector can be disposed in or on the body of a subject. For example, an automatic seizure detector, and other components of the intelligent brain pacemaker, can be disposed under the skin of a subject, making the intelligent brain pacemaker entirely self-contained within the body of a subject. Although the present invention discloses the use of a computer to visualize and store field potential data, the computer component can be omitted from the present invention and/or replaced with a simple digital storage device capable of transient data storage and any desired data processing. Additionally, radio telemetry-based components can also form an aspect of the present invention and can be employed to wirelessly transmit or download stored field potential data to a computer or other data storage or analysis component.

VI.B. Band-Pass Filter Module

The majority of activity occurring in the brain of a subject (as manifested by field potential voltage values) is normal and unrelated to seizure activity. Field potentials indicative of brain activity unrelated to seizure activity are typically of a constant signal with little change in voltage, as compared to activity during a seizure since, under non-seizure conditions, there is a low level of synchronization of field potentials and thus a minimal synergistic signal additive effect. In many circumstances, therefore, it is unnecessary to store and analyze these normal field potential measurements. Conversely, if field potentials are observed which appear to be higher or lower than normal, these field potentials with widely-ranging voltage levels might be a result of the hypersynchronized field potentials typically attendant with a seizure, it might be necessary to attenuate higher frequencies to facilitate the processing of these signals.

In one embodiment, a band-pass filter module of an automatic seizure detector can operate as a pre-filter to filter out frequency components of a signal that are extraneous to or could interfere with seizure detection. Such a filter can be applied in the present invention as a band-pass filter, with upper and lower cut-off values. A band-pass filter can be adapted to attenuate any frequency component of the signal that does not fall between these two values. Thus, frequencies, such as 60 Hz oscillations from nearby electrical devices, which could interfere with seizure detection, can be filtered out, whereas signal frequencies associated with a seizure will remain in the signal. This process generally makes the voltage signal from an electrode more representative of seizure activity, during the seizure detection process. Thus, a band-pass filter of the present invention preferably comprises a notch filter at 30 Hz, 60 Hz 90 Hz or another desired frequency.

A seizure detection module of an ASD device employs a signal that has been filtered by a band-pass filter in order to identify patterns of brain activity that characterize a seizure activity. Such a seizure detection module can employ any of a number of algorithms to identify a seizure. Such algorithms can be adapted to identify signals components such as the magnitude of the signal, the dominant frequency component of the signal, or the magnitude of the derivative of the signal in order to identify seizure activity, however this is not a complete list of signal components that can be employed in the present invention. When a seizure detection module detects seizure activity, it can issue one or more commands to a nerve stimulator, directing the nerve stimulator to stimulate the nerve with which it is associated.

VI.C. Seizure Detection Module

A seizure detection module of the intelligent brain pacemaker identifies seizure-related activity and, when such activity is identified, sends a signal that triggers electrical stimulation of a peripheral nerve. A seizure detection module thus accomplishes two broad functions: first, the module identification of a seizure and second, the module issues one or more stimulation commands to a nerve stimulator, directing the nerve stimulator to stimulate the nerve with which it is associated. A seizure detection module is preferably situated as a component of a single integrated unit, and more preferably the seizure detection module is integrated with the band-pass filter and other components automatic seizure detector components in a single module.

VI.C.1. Seizure Identification

One function of a seizure detection module is the identification of an occurring epileptic seizure. Preferably the seizure is identified as it is occurring, but more preferably the seizure is identified before it occurs. An algorithm can be employed to identify pre-seizure activity. As noted hereinabove, a hallmark of seizure-related brain activity is the appearance of signals comprising large changes in voltage values (i.e. spikes) in a field potential voltage profile. Such spikes can arise by hypersynchronization of brain activity and will quantitatively exceed those voltage measurements associated with normal, non-seizure related brain activity. Therefore, the presence of a seizure can be identified by the presence of voltage spikes in a field potential profile.

In one embodiment, a seizure detection module of an intelligent brain pacemaker detects the presence of a seizure by comparing incoming field potential data, which can be band-pass filtered to predetermined levels, with a predetermined threshold voltage value or other pattern of brain activity known to be associated with an epileptic seizure or condition. The seizure detection module can employ standard circuitry to analyze incoming data and make the comparison between the incoming data and the seizure detection algorithm.

An operator can preset the seizure detection algorithm. This seizure detection algorithm can be set manually either before or after the seizure detection module is situated in the final set up of the intelligent brain pacemaker. Appropriate seizure detection algorithms will be apparent to those of skill in the art upon consideration of the present disclosure. The precise seizure detection algorithm can be altered as desired. This ability facilitates the use of the intelligent brain pacemaker in conjunction with drug therapy, continued use of the intelligent brain pacemaker (which could have long term effects on the frequency of seizures) or other parameters that might affect the seizure detection algorithm over time.

A seizure detection algorithm can be a heuristic algorithm. That is, a seizure detection algorithm can be adapted to “learn” a subject's brain activity before, during and even after the occurrence of an epileptic seizure. Thus, in one aspect, a seizure detection algorithm can store one or more parameters which are monitored during an epileptic seizure of a subject. These data are then analyzed and stored. At a point in time after the seizure during which the data were taken, the seizure detection algorithm incorporates the data into the algorithm itself. Preferably, when a later seizure occurs or is predicted to occur, the seizure detection algorithm recognizes the onset of the seizure, based on measured data, and counteracts the seizure at an early point in time. Summarily, it is preferable that a seizure detection algorithm be adapted to evolve over time in such a fashion as to make the algorithm more effective at recognizing and preventing and/or ameliorating a seizure.

When a seizure detection module detects a signal that meets the requirements of the seizure detection algorithm, the module sends a signal to the nerve stimulator. This signal directs to the stimulator to deliver a stimulatory pulse or a series of stimulatory pulses to an electrode in contact with a cranial nerve, such as the trigeminal nerve. The nature and quality of the delivered pulse or pulses can vary and representative pulse schemes are described further hereinbelow.

It is preferable that a seizure detection module sends a signal to the nerve stimulator for a period of time equal to the period of time that the signal is found to meet the requirements of the seizure detection algorithm, and to not send signals to the stimulator when the seizure-related brain activity ceases. That is, stimulatory signals are only sent when a seizure is present and stimulatory signals are not sent when the field potential data falls below the threshold value or does not meet a known pattern of epileptic brain activity. By sending signals to the nerve stimulator only during periods in which seizure-related activity is present, side effects can be avoided. For example, by limiting the stimulation to the time when seizures are present or imminent, cardiovascular damage, which has been observed in vagus nerve systems, can be minimized or eliminated. Additionally, any potential damage or harm to the stimulated nerve can also be minimized or eliminated.

In one embodiment of the intelligent brain pacemaker, a TTL pulse is delivered to the nerve stimulator. A TTL circuit is a type of digital circuit in which the output is derived from two transistors. Thus, a TTL pulse is simply the digital signal output from a TTL circuit. The type and nature of signal delivered to the nerve stimulator will be determined by consideration of the components of the apparatus of the present invention, with the singular requirement that the nerve stimulator be activated upon detection of a signal meeting the requirements of the seizure detection algorithm.

VI.C.2. Stimulation is Provided Exclusively Before or During Seizure Activity

An advantage of the design of a data processor module of the present invention is that it can be configured to send a stimulation signal exclusively when a seizure is predicted or detected. This ability offers the beneficial therapeutic effect of removing the need to continually stimulate a nerve, thereby reducing the potential for nerve damage.

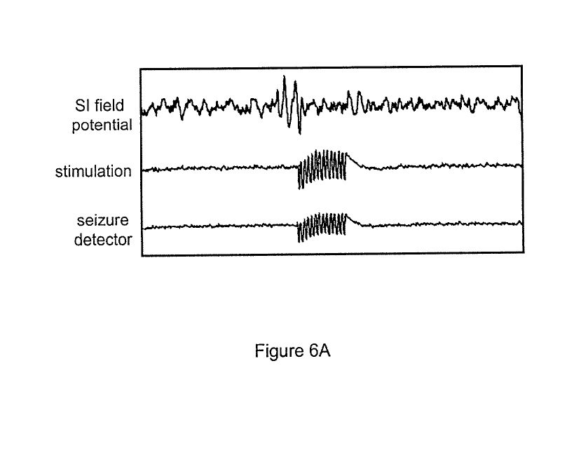

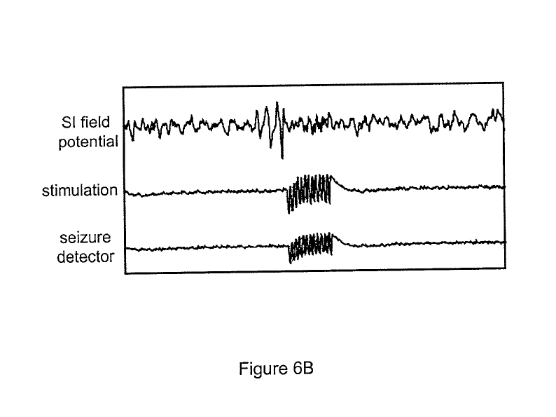

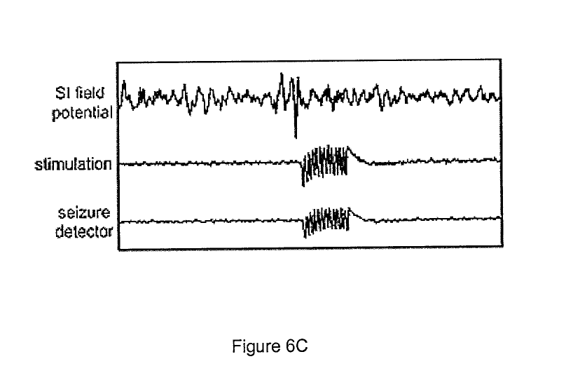

The efficacy of this design is demonstrated in FIGS. 6A-6C. In FIGS. 6A-6C, seizure-specific stimulation is seen to disrupt and stop hypersynchronous brain activity. FIGS. 6A-6C demonstrate that, when an observed field potential amplitude reaches a threshold value, the seizure detector triggers nerve stimulation, which eliminates the seizure, after which stimulation ceases. In FIGS. 6A-6C, the stimulation outlasts the seizure activity because pulses were provided in 500 msec trains. This operational parameter can be adjusted by an operator of the invention. Calibration for these figures is as follows: vertical, 200 μV; horizontal, 500 msec.

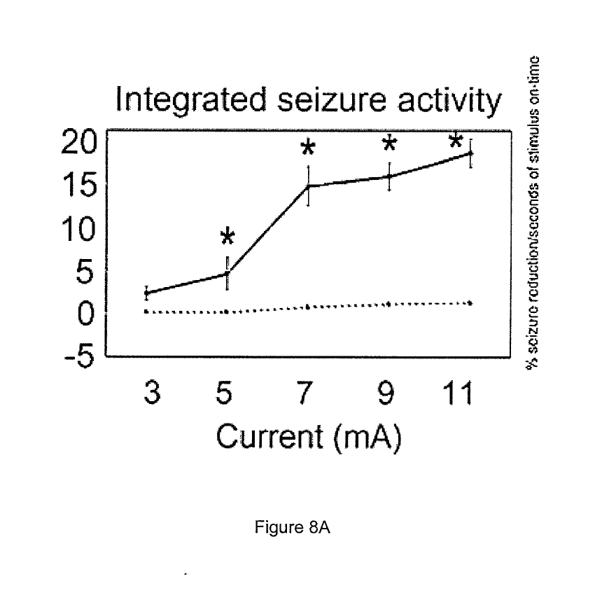

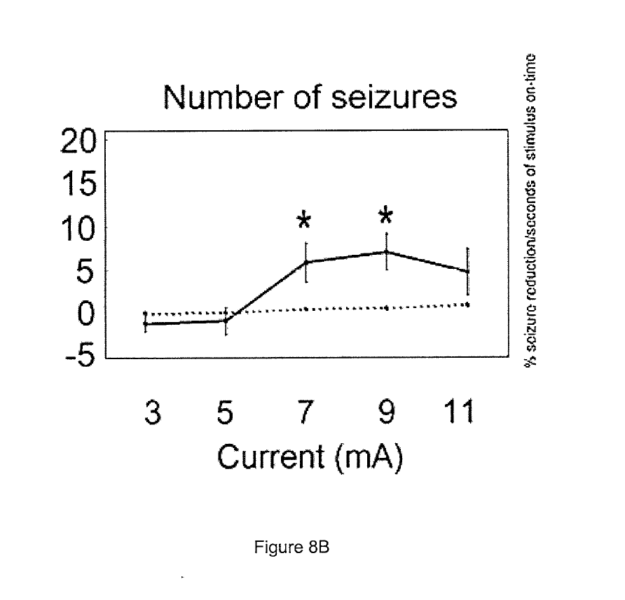

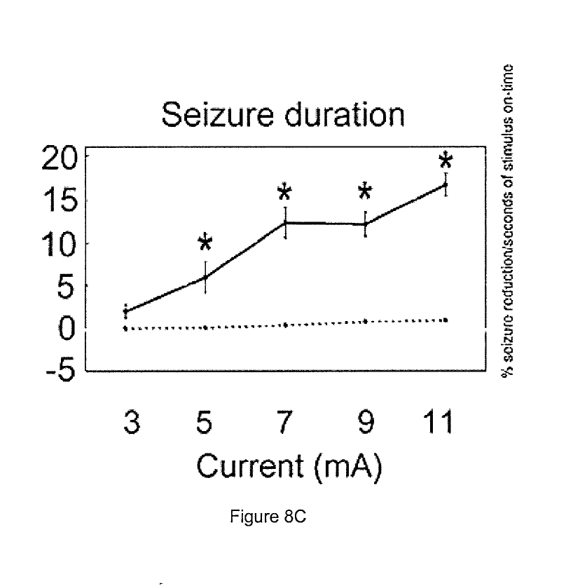

A direct comparison of the efficacy of regular periodic stimulation and stimulation only in the presence of a seizure further emphasizes this advantage over prior art methods and apparatuses. FIGS. 8A-8C depict a comparison of the amount of seizure reduction versus the amount of stimulation provided. Stimulation was provided both by the use of a periodic stimulation paradigm (i.e. regular periodic stimulation over a time interval, regardless of the presence of a seizure) and the automatic seizure detection aspect of the present invention (i.e. stimulation only when a seizure was detected). Stimulation provided by the periodic stimulation paradigm is represented in FIGS. 8A-8C by a dashed line, while stimulation provided by automatic seizure detection is represented by a solid line. The Y-axis represents the ratio of seizure activity reduction to seconds of stimulation in a given stimulus-on period. Asterisks designate the ratios of automatic seizure reduction to seconds of stimulation that were significantly higher than those obtained by the use of the periodic stimulation protocol. FIG. 8A depicts integrated seizure activity (calculated by summing all of the values for all of the amplitude range intervals for a given on or off period of stimulation); FIG. 8B depicts the number of seizures; and FIG. 8C represents seizure duration.

Comparing the ratios between ASD stimulation and periodic stimulation protocols depicted in FIGS. 8A-8C, it is evident that delivering stimulation only when seizure activity is detected is up to 39.8 times more effective at seizure reduction per second of stimulation than is periodic stimulation not correlated in any way to seizure activity. Thus, the intelligent brain pacemaker, which provides stimulation only during times of seizure, is significantly more effective at reducing seizures than other prior art methods which supply regular periodic stimulation.

Thus, a seizure detection module of the intelligent brain pacemaker can automatically detect the presence or onset of a seizure and send a signal to the nerve stimulator, triggering stimulation of the nerve with which it is associated. Stimulation ceases following the subsidence of the seizure. This ability obviates the need for prolonged or repetitive stimulation of a nerve, which is a component of prior art methods and apparatuses.

VII. Nerve Stimulator

In the fields of neurology and physiology, stimulators are generally employed to generate DC pulses according to a set of operator-specified parameters, which can include pulse amplitude and timing. Relevant timing parameters can include delay, duration, train duration and pulse interval. Delay is the time between single pulses, duration is the length of a single pulse, train duration is the time from the beginning of the first pulse of a train of pulses to the end of the last pulse of the train and pulse interval is the time between the pulses in a train of pulses. Additional parameters that can be controlled by the nerve stimulator include the frequency of the pulses that are delivered. It is preferable to employ a train of pulses in the intelligent brain pacemaker, although discrete pulses can be employed as circumstances dictate and at the discretion of the operator.

When a signal is received from the seizure detection module, the nerve stimulator executes a set of instructions corresponding to the type of nerve stimulation to be provided. The exact nature of an appropriate pulse scheme will be apparent to those of skill in the art upon consideration of the present disclosure, however one pulse scheme that can be employed comprises a 0.5 second pulse train of 500 μsec pulses delivered at 333 Hz.

Suitable nerve stimulators for practicing the intelligent brain pacemaker include the GRASS Model S8800 stimulator (available from Grass Instruments of Quincy, Mass.). A suitable nerve stimulator will be programmable, thereby allowing a wide range of pulse profiles to be created and delivered.

VII.A. Nerve Contact Electrode

The nerve stimulator generates and emits electrical pulses and pulse trains. The character of the pulses is preferably programmed into the nerve stimulator before pulses are emitted. These pulses and pulse trains are directed to the nerve that is to be stimulated (i.e. the trigeminal nerve). The interaction between the pulses generated by the nerve stimulator and the nerve to be stimulated is mediated by a nerve contact electrode, which is preferably a nerve cuff electrode. The nerve contact electrode can be a component part of the nerve stimulator or can be an additional component fitted to the architecture of the nerve stimulator.

The electrode in contact with the nerve to be stimulated is manufactured of a conductive material so as to transmit an electrical pulse. Additionally, the electrode is preferably treated to minimize any potential physiological reaction to the electrode, and to insulate the portion or portions of the electrode that does not contact the nerve. Suitable insulation materials include TEFLON® for a lead wire and SYLGARD® (available from Dow Corning Corp. of Midland, Mich.) for a nerve contact electrode.

In a preferred embodiment of a nerve contact electrode, the electrode is a nerve cuff electrode. A nerve cuff electrode is an electrode designed to encircle the nerve to be stimulated, thereby increasing the area of contact and stimulation. A suitable nerve cuff electrode can comprise one or more conductive bands optionally mounted on support surface. Preferably, a conductive band comprises platinum. When a plurality of conductive bands are employed in a nerve cuff electrode, it is preferable that the bands be communicatively associated with one another, such that when a stimulation pulse is applied to the nerve cuff electrode it is dispersed through all bands of the electrode. Additional wire or other material can be affixed to the nerve cuff electrode in order to permit its emplacement around the nerve to be stimulated. Areas of the electrode through which it is not desired to transmit voltage are coated with an insulator.

Leads from each band of the nerve contact electrode are attached to the nerve stimulator such that the stimulator, when activated, will pass current from one bad to the next. When current passes from one band to the next, this activates the nerve.

VII.B. Bilateral and Unilateral Stimulation and Implantation of Nerve Contact Electrodes

Nerve cuff and nerve contact electrodes can be implanted by surgically exposing the nerve and orienting the electrodes such that they surround the nerve. Turning first to nerve contact implantation strategies, nerve cuff and nerve contact electrodes can be implanted either on only one branch of a nerve, (e.g., the left branch of the IO nerve), or on both branches of the nerve (e.g., the right and left branches of the IO nerve). Implantation of a nerve cuff electrode on a single branch of a nerve present in a subject can facilitate unilateral stimulation of that nerve. However, implantation of a nerve cuff electrode on two or more branches of a nerve present in a subject can facilitate bilateral stimulation of that nerve.

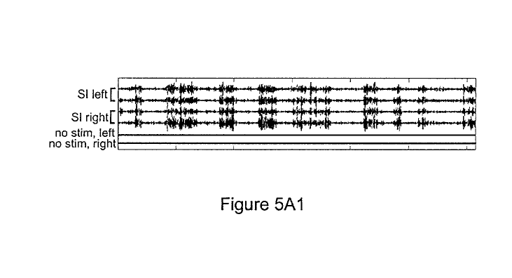

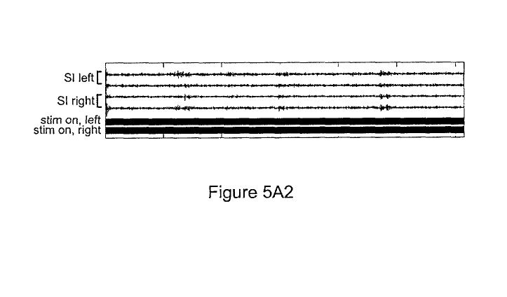

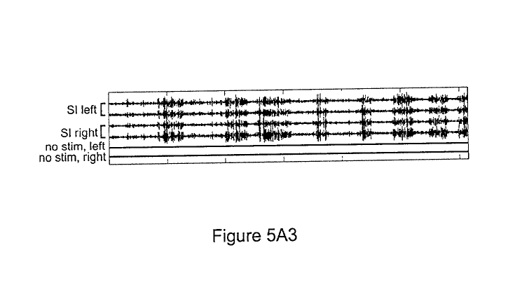

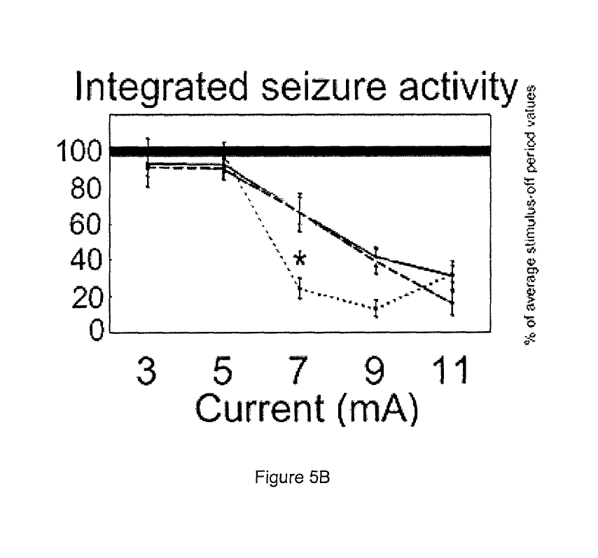

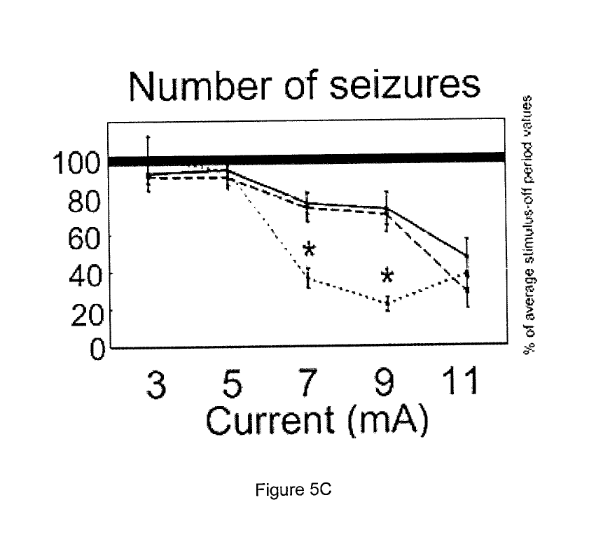

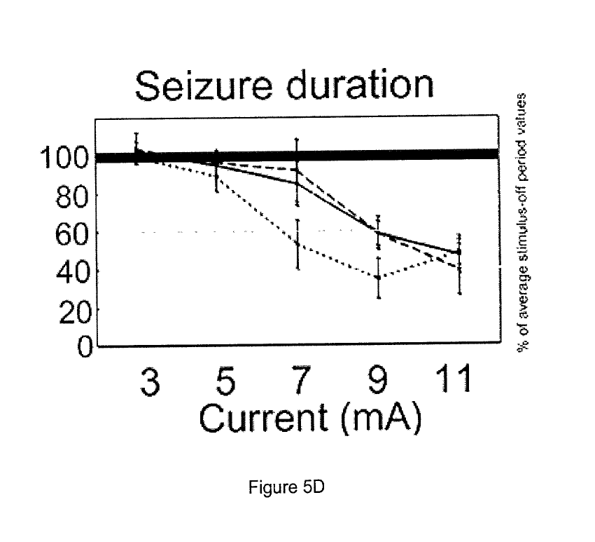

The choice of unilateral or bilateral implantation and stimulation can affect the amount of stimulation required for stimulation reduction. By way of example, FIG. 5 depicts the effects of bilateral stimulation versus unilateral stimulation of the IO nerve. FIGS. 5A1-5A3 depict filtered field potential traces showing seizure activity during three sequential one minute periods. FIG. 5A1 depicts no stimulus applied; 5A2 depicts application of bilateral stimulation; and 5A3 depicts no stimulus applied. The stimulus parameters giving rise to the traces depicted in FIGS. 5A1-5A3 are 9 mA, 333 Hz and a 0.5 msec pulse duration. FIGS. 5B-5D depict average values presented as ratios of stimulus on to stimulus off measurements. FIG. 5B depicts integrated seizure activity, FIG. 5C depicts the number of seizures and FIG. 5D depicts seizure duration. In FIGS. 5B-5D, a solid line connects responses contralateral to the simulation site, a line with long dashes connects responses ipsilateral to the stimulation site and a line with short dashes connects responses to bilateral stimulation. Responses to bilateral stimulation that are significantly different (based on a statistical evaluation of the data) from those to ipsilateral and contralateral stimulation are represented by an asterisk. Error bars represent +/− SEM.

FIGS. 5A-5D demonstrate that bilateral stimulation is equally effective as is unilateral stimulation, but require less current to do so. Importantly, this observation demonstrates that the use of bilateral stimulation of a nerve can decrease the amount of current required to reduce a seizure. The use of lower currents to achieve seizure reduction, can lead to decreased nerve damage, since less current will actually interact with the nerve, as well as a reduction in side effects that might occur during stimulation. Therefore, when implanting a nerve cuff electrode or a nerve contact electrode, it might be desirable to implant a plurality of these electrodes in a subject so as to decrease the level of current required to counteract a seizure. Thus, nerve cuff electrodes and nerve contact electrodes can be implanted with the goal of either unilateral or bilateral nerve stimulation.

It is noted, however, that although bilateral stimulation can reduce the level of required current, the intelligent brain pacemaker can function equally well by employing unilateral stimulation. The precise number, location and strategy for implanting a nerve cuff electrode or a nerve contact electrode, then, can be dictated in part by the subject's physiology and the judgment of the individual making the implantation.

VII.C. Nerve Stimulation Parameters

The character and degree of the stimulation provided to a nerve can have an effect on the efficacy of the stimulation. It is therefore preferable for an operator to optimize the characteristics of the stimulation pulse. For example, if a stimulation pulse train is employed to stimulate a nerve, the composition and amplitude of the pulse train can be considerations. Optimization of nerve stimulation parameters can enhance the efficacy of nerve stimulation.

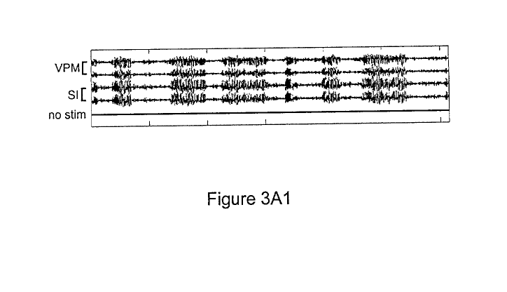

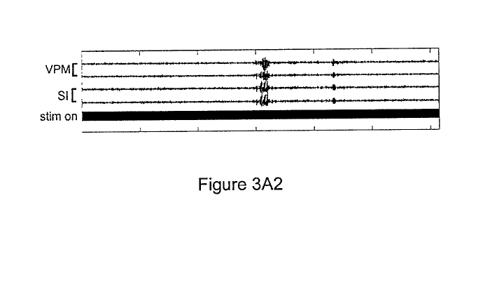

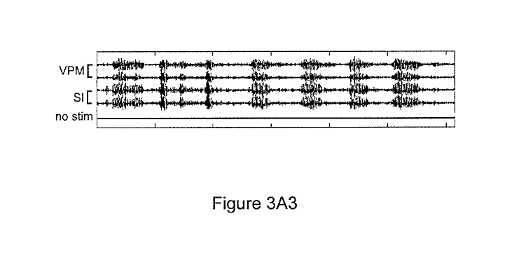

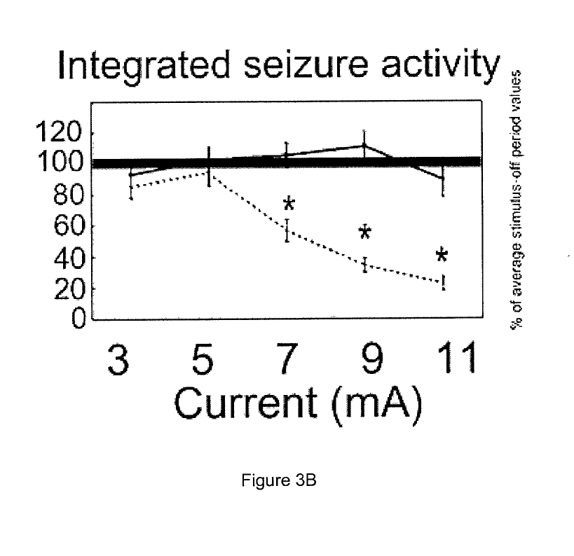

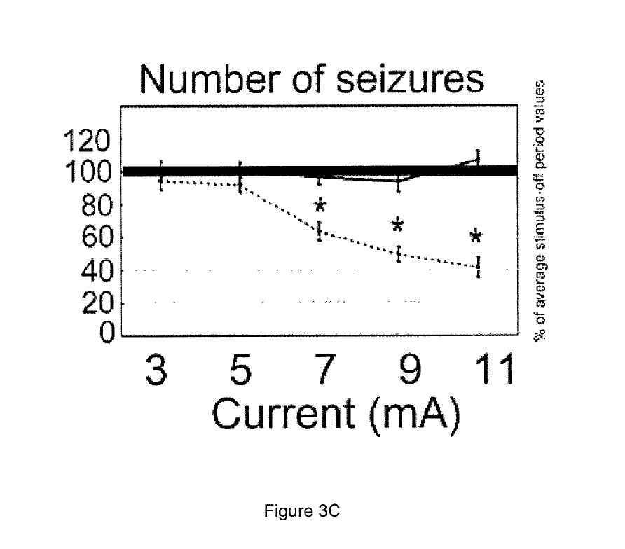

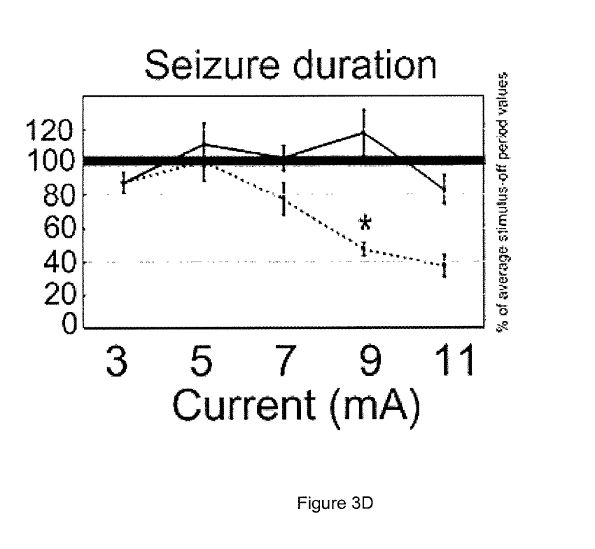

FIGS. 3A-3D depict results of the stimulation of the IO branch of the trigeminal nerve and indicate that stimulation of the IO nerve reduces seizure activity in a current dependent manner. In FIGS. 3A1-3A3, filtered field potential traces showing seizure activity during three sequential one minute periods are depicted. The parameters for the stimulatory pulses giving rise to the data presented in these figures were 11 mA 333 Hz and a 0.5 msec pulse. In FIGS. 3B-3D, the amount of seizure activity during one minute periods of stimulation at different current levels compared with the period of no stimulation directly preceding each stimulus-on period. Values are presented as a percent of the average stimulus-off period measurements. FIG. 3B represents the effect of stimulation on integrated seizure activity; FIG. 3C represents the effect of stimulation on the number of seizures observed; and FIG. 3D represents the seizure duration. Error bars represent +/− SEM. Solid lines connect stimulation-off values; a dashed line connects stimulation-on values. An asterisk designates stimulation-on values that are significantly different from stimulation-off values in these figures. Thick black horizontal lines at 100% denote the level of no change in seizure activity.

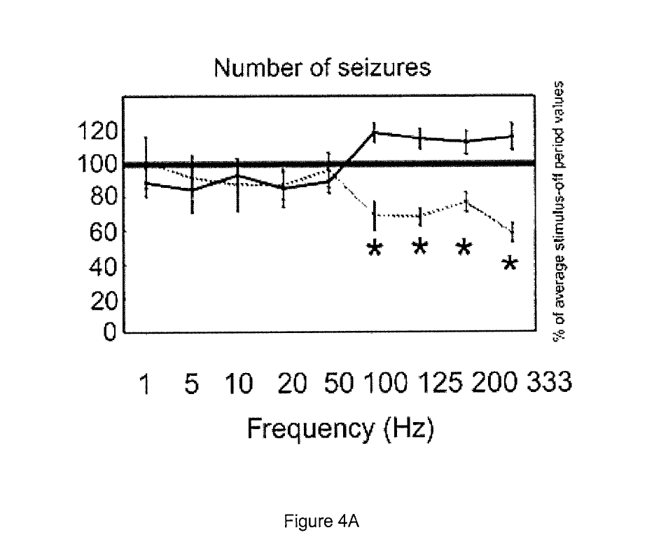

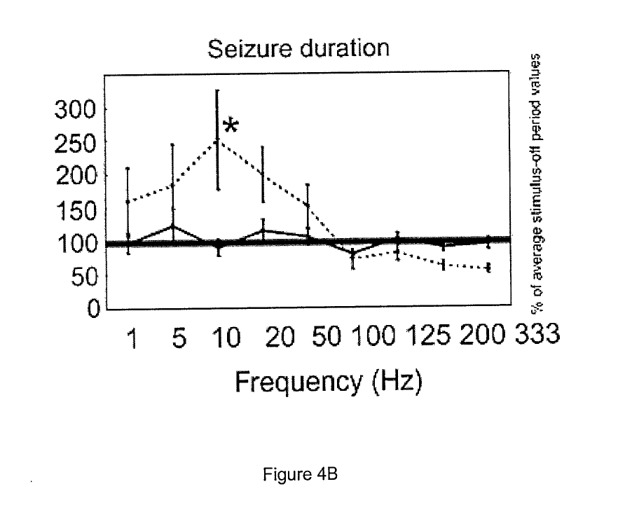

The plots depicted in FIGS. 4A and 4B demonstrate the effect of varying the stimulus frequency using the periodic stimulation paradigm. In other words, these figures indicate that the stimulus frequency is also a stimulation parameter which can be optimized. The calibration and general description of FIG. 3 is applicable to FIG. 4. That is, error bars represent +/− SEM. Solid lines connect stimulation-off values; a dashed line connects stimulation-on values. Stimulation-on values significantly different from stimulation off values are designated by an asterisk in the figures. Thick black horizontal lines at 100% denote the level of no change in seizure activity. FIG. 4A demonstrates the effect of varying the stimulus frequency on the number of seizures, and FIG. 46 demonstrates the effect of varying the stimulus frequency on the duration of the seizures.

Summarily, FIGS. 3A-3D demonstrate that stimulation of the IO nerve decreases seizure activity overall, in a current dependent manner. FIGS. 4A and 4B demonstrate that the stimulation frequency can have an effect on the efficacy of nerve stimulation. Therefore, stimulation current and frequency, which are preferably set or programmed into the nerve stimulator, are two stimulation parameters that can be optimized. Other stimulation parameters that can be optimized include pulse duration, pulse frequency and the duration of a stimulus pulse train.

VIII. Data Analysis

As disclosed hereinabove, it might be desirable to perform an analysis of the seizure-related data acquired by the intelligent brain pacemaker. For example, it might be desirable to characterize or quantify seizure activity by seizure frequency, seizure duration, and/or integrated seizure activity. By employing software purchased or written for this purpose, this type of data analysis can be performed. Additionally, a statistical analysis can be performed in order to quantitatively assess the significance of acquired data.

VIII.A. Mathematical Characterization of Seizure Data

In an example of a type of data analysis that can be performed, field potential traces can first be band-pass filtered at 5-30 Hz. A sliding window (e.g., a 1 sec window with a 0.5 sec overlap) can then be used to quantify the activity of the absolute values of the field potential traces. Within each window, the amplitude (i.e. voltage) range of the absolute value of the field potential activity in each trace can be subdivided into equal parts, and within each sliding window the number of voltage values falling into each of the divisions of the amplitude range can then be calculated. A threshold value, for example 50% of the amplitude range, can then be used to identify seizure activity. If activity within a predetermined number of consecutive windows is above this threshold, the activity can be considered to be part of a seizure. From these data, the number of seizures and their durations can then be calculated by counting the number of windows during which activity was above the threshold. In addition, a measure denoted the “integrated seizure activity” can be calculated by summing all of the values for all of the amplitude range intervals for a given “on” or “off” period of stimulation. This algorithm can be applied in a uniform, blinded manner to all data, allowing for an objective quantification of seizure activity. This type of mathematical data treatment can be performed by a seizure detection module, if the module is so configured.

VIII.B. Statistical Analyses of Seizure Data

It might be desirable to perform a statistical analysis of data acquired from a subject. Such an analysis can be performed as an aspect of a research project, or can be performed on a subject in which an intelligent brain pacemaker is implanted, in order to statistically evaluate calculated measurements of seizure frequency, seizure duration, and/or integrated seizure activity. A variety of statistical analyses can be performed, and the nature of the analysis will depend in part upon the type of data available. When quantitative seizure-related data is available, such as seizure frequency, seizure duration, and/or integrated seizure activity, the following general approach can be employed.

Using the values for seizure number, seizure duration, and integrated seizure activity, the efficacy of IO nerve stimulation and seizure detection can be assessed by comparing each stimulation-on period with the stimulation-off period directly preceding it. Thus, results of this comparison can be presented as ratios of seizure activity during stimulus-on periods to seizure activity during stimulus-off periods. Multivariate ANOVAs (MANOVAs) can be employed to assess whether there are statistically significant changes in seizure duration, seizure frequency, or integrated seizure activity between periods of no stimulation and periods of stimulation for each stimulus parameter setting. In addition, repeated measure ANOVAs can be employed when comparing one measure with another (e.g., number of seizures compared with seizure duration). When significant differences are indicated by MANOVA or ANOVA analyses, Tukey's honestly significant difference post hoc tests can be employed to identify which effects were significant, which can be based on a calculated p score of <0.05.

IX. An Implantable Self-contained Intelligent Brain Pacemaker for Human Epilepsy Patients

The intelligent brain pacemaker need not employ a computer to visualize the field potential data. This component can be omitted. When this bulky component is omitted, an intelligent brain pacemaker can be configured to be integrated and entirely self-contained. This embodiment is suitable for use by human patients and can be employed by the patient continuously in day-to-day life, thereby greatly reducing the number, length and severity of epileptic seizures and increasing the patient's quality of life. The intelligent brain pacemaker can be configured similar to the cardiac pacemakers currently available and can operate as continuously and inconspicuously as these common cardiac pacemakers do.

The component parts of an integrated, self-contained intelligent brain pacemaker can be situated entirely within the body of a patient. Microwire electrodes can be on the order of 50 μm in diameter, making them convenient to chronically implant in the brain tissue of a patient. Alternatively, electrodes can be placed subdurally, under the scalp of a patent or on the surface of a patient's skin. Proper placement and/or emplacement techniques can be selected to generate a minimum of patient discomfort. The electrodes should be well insulated, which can be accomplished by coating the leads with an insulating biomaterial or an insulator such as polytetrafluoroethylene.

The seizure detector can comprise a small computer chip or wafer. The size of the detector will be dictated primarily by design considerations for the detector circuitry. Given the recent advances in chip technology, it is now possible to design and build a seizure detector of microscale or nanoscale dimensions. The small size of the seizure detector allows it to be implanted at a range of locations in or on the body of a patient. For example, a seizure detector can be placed beneath the skin of the patient's scalp, neck or chest. The small size of the microwire electrodes permits these electrodes to easily pass from the region of implantation to the seizure detector. In one embodiment, the seizure detector can be placed under the scalp of a patient and the electrodes can run from their emplacement in the patient's brain tissue through apertures in the skull to the seizure detector.

It is also preferable to situate the stimulator within the patient's body. Although the laboratory scale stimulator is too bulky to be realistically considered for implantation within the body of a patient, smaller stimulators can be employed. For example, the generator employed in a cardiac pacemaker is routinely implanted under the skin beneath the collarbone of a patient. A stimulator of similar dimensions can be employed as a component of the intelligent brain pacemaker, and can be positioned in roughly the same location. Typically, an enclosed lithium battery typically powers a cardiac generator. The intelligent brain pacemaker can also employ a sealed lithium battery to power the stimulator, which can power the intelligent brain pacemaker for years at a time. The intelligent brain pacemaker can be configured such that replacement of a spent power source can be performed by making a small incision in a subject, removing any associated attachment wires and simply attaching a fresh power source.

In another aspect of the present invention, a nerve stimulator situated under the skin of a patient can be configured to permit it to be programmed, using a device held over the skin of a patient that is located over the stimulator itself. The seizure detector can similarly be configured to respond to an external programming device in the same fashion. In this configuration, the threshold voltage, pulse characterization and other operational parameters can be modified without performing additional surgery. This embodiment of an intelligent brain pacemaker implanted in the body of a patient can operate entirely unattended, and additional surgery need only be performed when the stimulator power supply needs to be replaced.

From time to time, it will be preferable to make periodic checks on the operational status of the intelligent brain pacemaker. When the device is implanted in the body of a patient, the device can be inspected by surgically removing the device and performing a series of diagnostic checks on the invention. However, this approach requires that the patient to undergo additional surgery. To circumvent the need for additional surgery, the component parts of the intelligent brain pacemaker can be configured to permit diagnostic checks to be performed over the telephone, as diagnostic checks on cardiac pacemakers can be presently made. Specifically, the seizure detector and/or the nerve stimulator can be configured to transmit a signal to a device placed on the skin above the implanted component parts of the present invention. The device can then relay the signal over a telephone line to a computer at a remote location, such as a physician's office.

X. Advantages of the Intelligent Brain Pacemaker Over the Prior Art

The present invention offers a range of advantages over epilepsy treatments known in the art. Several advantages have been discussed hereinabove, such as the ability to implant the invention entirely within the body of a patient and the fact that operation of the intelligent brain pacemaker is automatic and can function without human intervention (i.e. manual triggering of electrical pulses). Some, but not all, advantages of the present invention are disclosed hereinbelow.

X.A. Nerve Stimulation Only During Times of Seizure Reduces Damage to the Stimulated Nerve

One advantage of the present invention is that triggering nerve stimulation only when a seizure is occurring, or just prior to the onset of a seizure, is a much more effective method for reducing seizure activity than is providing stimulation on a fixed duty cycle, which has been employed in the prior art. This advantage represents an important advancement in the use of cranial nerve stimulation therapies in epilepsy for several reasons.

One advantage of stimulation of a cranial nerve not associated with an autonomic function only during times of seizure is that this ability can, for many patients, reduce the overall amount of stimulation required for maintaining seizure control. Thus, the amount of potentially unnecessary stimulation usually occurring between seizure periods is reduced, decreasing the possibility of damage to the nerve (Agnew et al., (1 989) Ann. Biomed. Eng. 17: 39-60; Agnew & McCreery, (1990) Epilepsia 31 [Suppl. 2]: S27-S32; Ramsay et al., (1994) First International Vagus Nerve Stimulation Study Group. Epilepsia 35: 627-636). Additionally, it has been demonstrated that there is a prophylactic effect associated with vagus nerve stimulation such that after stimulation, seizures are less likely for a period of time related to the duration of the preceding stimulation (Zabara, 1992 Epilepsia 33:1005-1012; Takaya et al., 1996 Epilepsia 37: 1111-1116). This implies that an optimal overall treatment stimulation protocol involves the use of seizure-triggered stimulation combined with intermittent prophylactic nonseizure-triggered stimulation.

X.B. Nerve Stimulation Only During Times of Seizure Reduces Side Effects of Nerve Stimulation

Another advantage of the present invention is that it can reduce the side effects experienced by patients when the stimulus is on. For example, patients undergoing VNS treatment report hoarseness, coughing, and throat pain as the most common side effects of the stimulation (Ramsay et al., (1994) First International Vagus Nerve Stimulation Study Group. Epilepsia 35: 627-636; McLachlan, (1997) J. Clin. Neurophysiol. 14: 358-68; Schachter & Saper, (1998) Epilepsia 39: 677-686). These side effects are generally only experienced when the stimulation is on. However, if stimulation is presented only in response to the detection of seizure activity (or occasionally prophylactically, as described above), these side effects are minimized.

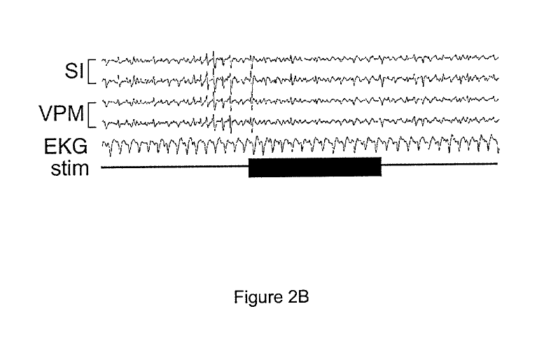

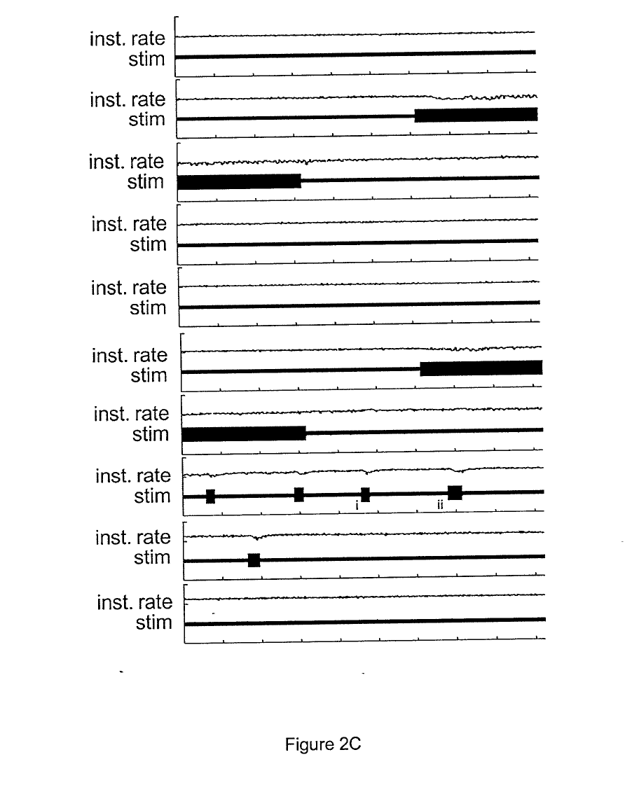

Additionally, the intelligent brain pacemaker minimizes the risk of cardiovascular damage, which is a consideration in VNS therapy. FIGS. 2A-2C are EKG traces that indicate that EKG activity is not significantly altered during IO nerve stimulation. FIGS. 2A and 2B are two examples of EKG activity during IO nerve stimulation in an anesthetized rat. Calibration for these figures is as follows: vertical 100 μV and horizontal 1 sec. FIG. 2C depicts the EKG traces and instantaneous heart rate over a 15 minute period during which stimulation was twice provided continuously for 1 minute as well as five times for shorter bursts. Small changes in the EKG can be seen when stimulation is provided, but they are minor and rapidly stabilize, even during ongoing stimulation. Roman numerals i and ii are traces that are shown at a faster time scale in FIG. 2A and FIG. 2B. Calibration for FIG. 2C is as follows: vertical, 100 μV for the EKG traces, 200 beats/min for the instantaneous heart rate; horizontal, 10 seconds. The stimulus parameters in these trances are 50 Hz, 11 mA and a 0.5 msec pulse duration.

These figures demonstrate that there are no significant cardiac-related side effects associated with stimulation of a cranial nerve not associated with autonomic function, such as the IO branch of the trigeminal nerve. This advantage is absent from other prior art methods, which can cause serious and permanent cardiac damage as a result of stimulation of a cranial nerve associated with an autonomic function, such as the vagus nerve, or other structure.

X.C. The Intelligent Brain Pacemaker Can be Implemented in Humans

Yet another advantage of the present invention is that the real-time, automatic seizure detector described herein can be implemented in humans. It is important to note that the intelligent brain pacemaker is not simply a research tool and the invention has significant clinical relevance. Often, treatments effective in laboratory environments cannot be practically implemented to benefit humans. Therefore, a significant advantage of the intelligent brain pacemaker is that it can be implemented in humans to detect and reduce seizures and the effects of seizures.

When the intelligent brain pacemaker is situated in a human, as described hereinabove, seizure detection can be performed by a computer microchip programmed with a seizure detection algorithm (the seizure detector) and carried by the patient, similar to the HOLTER monitors used for continuous EKG monitoring. In one embodiment, input can be delivered to this microchip from multiple scalp EEG electrodes that are able to pick up and amplify extracranial EEG signals. When the microchip detects seizure activity in the EEG signals, it can trigger an implanted stimulator, which then stimulates one or more nerve contact electrodes. In this way, the intelligent brain pacemaker functions in a manner analogous to cardiac pacemakers, commonly used to treat heart arrhythmia, and requires a minimum of invasive procedures. In essence, this device constitutes a “brain pacemaker” for seizure monitoring and control.

X.D. The Intelligent Brain Pacemaker Incorporates Reliable Seizure Detection Methods That Can Also Predict Seizures

The application of nonlinear computational methods for detection of seizure activity (Gabor et al., (1996) Electroencephalogr. Clin. Neurophysiol. 99: 257-266; Webber et al., (1996) Electroencephalogr. Clin. Neurophysiol. 98:250-272) can be extremely beneficial when incorporated into the seizure detector described in the present disclosure. Such seizure detection algorithms allow for very accurate identification. Another substantial advantage in the implementation of the seizure prediction algorithms of the present invention is that this approach can identify seizures seconds or minutes before the behavioral onset (See generally Martinerie et al., (1998) Nat. Med. 4:1173-1176; Le Van Quyen et al., (1999) NeuroReport 10: 2149-2155).

There is evidence that the sooner stimulation is provided after a seizure begins, the more effectively the seizure can be stopped (Uthman et al., (1993) Neurology 43: 1338-1345); also stimulation is more likely to prevent seizure activity if it is presented before, rather than after a seizure has begun (Woodbury & Woodbury, (1990) Epilepsia 31 [Suppl. 2]: S7-S19). Therefore, it is preferable to initiate simulation by employing the present invention before the clinically defined onset of a seizure can prevent seizures before they begin or become behaviorally relevant to the patient. The seizure detector and nerve stimulator can be programmed to respond in this way. As more sophisticated seizure detection and prediction algorithms are developed, these algorithms can be implemented in the intelligent brain pacemaker, which offers the advantage of programmability. Thus, the present invention can dramatically improve the efficacy of the cranial nerve stimulation therapy.

XI. Conclusion

The intelligent brain pacemaker demonstrates a range of substantial advances in the use of cranial nerve stimulation for the treatment of seizures over treatments known in the art. For example, the present invention relies on stimulation of a cranial nerve not associated with an autonomic function, such as the trigeminal nerve; prior art methods and apparatuses rely instead on stimulation of cranial nerves associated with an autonomic function, such as the vagus nerve. Data demonstrating the efficacy of reducing PTZ-induced seizure activity in rats is presented in the figures and Laboratory Examples set forth hereinafter. The present invention, therefore, can ameliorate seizures by stimulation of a cranial nerve other than the vagus nerve, which is the basis of several prior art methods.

Additionally, bilateral stimulation of a cranial nerve not associated with an autonomic function, an aspect of the present invention, can have the same seizure reduction effect as unilateral stimulation but requires much less current to do so. In fact, bilateral stimulation of a cranial nerve associated with an autonomic function is not a viable option, since this practice can endanger the health of a subject. This aspect of the present invention is therapeutically relevant, because multi-site stimulation can help maximize the seizure reduction effect of the present invention and reduce side effects that might occur with nerve stimulation, while employing the lowest current levels possible.

Moreover, automatic real-time seizure-triggered stimulation reduces seizures more effectively, per second of stimulation, than does periodic stimulation that is unrelated to seizure onset, such as those techniques known in the art. Therefore the use of the real-time brain-device interface of the present invention that automatically detects seizure activity and triggers a nerve stimulator only when such activity was present provides a high degree of seizure control, while potentially reducing the overall amount of stimulation presented to a patient. This aspect of the intelligent brain pacemaker offers a significant improvement in the efficacy of cranial nerve stimulation as a therapy for patients with intractable epileptic seizures.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Materials and Methods for Laboratory Examples 1 to 5

The following materials and methods were employed in Laboratory Examples 1 to 5. The results of the Examples are discussed immediately following the Laboratory Examples.

### Subjects

Eight adult female Long-Evans hooded rats weighing between 230 and 375 gm served as subjects in this study. All procedures and experiments were conducted in compliance with Duke University Medical Center animal use policies and were approved by the Duke University Institutional Animal Care and Use Committee.

### Induction of Seizures

Seizures were induced by intraperitoneal injection of PTZ (40 mg/kg). This dose of PTZ induced generalized seizure activity for 1-2 hr. This seizure activity was manifested in two ways: (1) highly synchronous, large-amplitude activity in the thalamic and cortical field potential traces (which are depicted in FIGS. ((2, 3, 5-7)) and (2) clonic jerking of the body and forelimbs. These two indicators of seizure activity were highly correlated at all times, as assessed by concurrent visual inspection of the animal and the real-time field potential traces. Occasionally, a supplemental dose of PTZ (7-10 mg/kg) was given if seizures ceased in <1 hr.

### Nerve Cuff Electrodes

The infraorbital nerve (or nerves) was stimulated unilaterally or bilaterally via chronically implanted nerve cuff electrodes. These electrodes were constructed in-house and consisted of two bands of platinum (0.5 mm wide and 0.025 mm thick; ˜0.8 mm separation between bands) that ran circumferentially around the infraorbital (IO) nerve, and are depicted in FIG. 1. The platinum bands were held in place and electrically insulated by a thin SYLGARD® (available from Dow Corning, Inc. of Midland Michigan) coating. Each band was connected to a piece of flexible, three-stranded TEFLON® (available from DuPont Co. of Wilmington, Del.) coated wire that was used to pass current between the bands (Fanselow & Nicolelis, (1999), J. Neurosci. 19: 7603-7616).

### Chronic Implantation of Microwire Electrodes

Microwire electrodes (available from NBLabs of Denison, Tex.) were chronically implanted into the ventral posterior medial thalamus (VPM) and/or primary somatosensory cortices (SI) for use in recording field potentials in these areas, as depicted in FIG. 1. Three rats had arrays of 16 microwires implanted in layer V of the SI cortex and bundles of 16 electrodes implanted into the VPM thalamus, both contralateral to the stimulated nerve. Five rats had two arrays of 16 electrodes implanted, one each into layer V of the left and right SI cortices so that recordings could be made both ipsilateral and contralateral to the nerve being stimulated. These implants were performed under pentobarbital anesthesia (50 mg/kg). Small craniotomies were performed over the areas into which electrodes were to be implanted (coordinates from Paxinos & Watson, (1986) The Rat Brain, Ed. 2. New York: Academic, Harcourt, Brace and Jovanovich). Electrodes were slowly lowered into these areas, and recordings were made throughout the implantation process to assess electrode location. After electrodes were in the correct position, they were cemented to skull screws by the use of dental acrylic (Nicolelis et al., (1997) Neuron 18: 529-537).

### Chronic Implantation of Nerve Cuff Electrodes

After implantation of the microwires, nerve cuff electrodes were implanted either unilaterally or bilaterally. A dorsoventral incision was made on the face several millimeters caudal to the caudal edge of the whiskerpad. Tissue was dissected until the infraorbital nerve was exposed, and the cuff electrode was positioned around the nerve such that the nerve lay inside the cuff. The cuff was then tied around the nerve to hold it in place, and the wound was sutured. The TEFLON® (available from DuPont Co. of Wilmington, Del.) coated leads from the platinum bands were run subcutaneously to the top of the head where they were attached to connector pins and affixed to the skull.

### Recording Procedures

Field potential recordings from VPM thalamus and SI cortex were made using chronically implanted microwires (Nicolelis et al.,(1997) Neuron 18: 529-537). Field potentials were collected using a GRASS Model 15 amplifier and stored on a personal computer. Signals were collected at a sampling rate of 512 Hz and band pass filtered during collection at 1-100 Hz. During each recording session, 16 field potential channels were recorded, 8 from each area from which recordings were made in a given rat (either VPM and SI, or SI left and SI right). In addition, one channel was recorded for each nerve cuff being stimulated (unilateral or bilateral stimulation) to indicate when stimulation occurred. During experiments, animals were awake and allowed to move freely in a 30 cm×30 cm recording chamber.

### Stimulation Parameters

Stimulation of the 10 nerve cuff electrodes was provided by the use of a GRASS S8800 stimulator (available from Grass Instrument Co of Quincy, Mass.) in conjunction with a GRASS SIU6 (available from Grass Instrument Co of Quincy, Mass.) stimulus isolation unit. Unimodal square current pulses with a duration of 500 μsec were given at a range of currents and frequencies. Current values were varied from 3 to 11 mA (2 mA intervals), and frequency values were varied from 1 to 333 Hz (1, 5, 10, 20, 50, 100, 125, 200, and 333 Hz). Animals tolerated stimulation at these levels without indication of pain, although in some animals there appeared to be a sensation of pressure on the face at the highest current and frequency settings. This was evidenced by a tendency for the animals to back up when the stimulus began, in the direction away from the stimulated side if unilateral stimulation was provided or straight back in the case of bilateral stimulation. In addition, at lower stimulus intensities animals would occasionally scratch at the whiskerpad on the side of the face being stimulated during the first few seconds of stimulation. However, the scratching was neither intense nor prolonged.

### Automatic Seizure Detection Device

The device shown in FIG. 1 and discussed hereinabove was designed and built in-house to automatically detect seizure activity in real time and immediately trigger a stimulator when a seizure was detected. The automatic seizure detection device (ASD) first low-pass filtered the raw field potentials obtained from the microwire arrays at 30 Hz. Circuitry then determined whether the field potential activity surpassed a threshold voltage value, indicative that seizure activity was present. When the field potential voltage crossed the threshold, a TTL pulse was sent to the GRASS S8800 stimulator (available from Grass Instrument Co of Quincy, Mass.), which delivered a 0.5 sec train of 500 μsec pulses at 333 Hz. The current level was dictated by the stimulation protocol for a given trial. Trains of stimuli were presented as long as the field potential activity remained above the threshold value (i.e., as long as seizure activity was ongoing). The train duration for the seizure-triggered stimulation (0.5 sec) was chosen because it was the shortest duration that found to be effective for stopping the seizure activity, and it was preferable to keep the stimulation as short as possible to reduce the total amount of stimulation given. The voltage threshold was set manually for each experiment. Generally, the seizure activity was three to five times that of the background activity, and the threshold was set high enough to identify seizure activity only. After the threshold was set for a given experiment, it was not moved. The seizure activity recorded on the field potential traces was directly correlated with behavioral manifestation of the seizures (clonic jerking of the body and forelimbs). When setting the seizure detection threshold, we always verified that the seizure activity identified by the ASD device was directly correlated with this behavioral component of the seizures.

### Experimental Protocols

The first part of this study was performed to determine whether stimulation of the IO branch of the trigeminal nerve was capable of eliminating PTZ-induced seizure activity in awake rats. To do this, we delivered continuous stimulation to the IO nerve during episodes of PTZ-induced seizure activity via the nerve cuff electrode (depicted in FIG. 1) for 1-minute stimulus-on periods, separated by 1 min stimulus-off periods. This protocol was performed with both unilateral and bilateral stimulation of the IO nerve. Stimulus parameters were varied between the stimulus-on periods as described above.

In the second part of this study, the effectiveness of stimulating the IO nerve was assessed only when seizure activity was present by using the ASD. For this protocol, the ASD device was turned on for 1-minute stimulus-on periods, separated by 1-minute stimulus-off periods, as in the first protocol, but stimulation was only provided during the stimulus-on periods when seizure activity was detected by the ASD device.

### Data Analysis

Seizure activity in the field potential recordings was measured in three ways: seizure frequency, seizure duration, and integrated seizure activity. These parameters were quantified by the use of a custom-made analysis program developed using MATLAB® (available from The Mathworks, Inc. of Natick Mass.). The field potential traces were first bandpass filtered at 5-30 Hz. A sliding window (1 second window with 0.5 sec overlap) was used to quantify the activity of the absolute values of the field potential traces. Within each window, the amplitude (i.e., voltage) range of the absolute value of the field potential activity in each trace was divided into 10 equal parts, and within each sliding window the number of voltage values falling into each of the 10 divisions of the amplitude range was calculated. Then, a threshold of 50% of the amplitude range was used to identify seizure activity. If activity within three consecutive windows was above this threshold, the activity was considered to be part of a seizure. From these data, the number of seizures and their durations could be calculated by counting the number of windows during which activity was above the threshold. In addition, a measure denoted the “integrated seizure activity” was calculated by summing all of the values for all of the amplitude range intervals for a given on or off period of stimulation. This algorithm was applied in a uniform, blinded manner to all of our data, allowing for objective quantification of the three measures of seizure activity.

### Statistical Analyses

Using the values for seizure number, seizure duration, and integrated seizure activity, the efficacy of IO nerve stimulation and seizure detection was assessed by comparing each stimulation-on period with the stimulation-off period directly preceding it. Thus, results are presented as ratios of seizure activity during stimulus-on periods to seizure activity during stimulus-off periods. Multivariate ANOVAs (MANOVAs) was employed to assess whether there were statistically significant changes in seizure duration, seizure frequency, or integrated seizure activity between periods of no stimulation and periods of stimulation for each stimulus parameter setting. In addition, repeated measure ANOVAs were used when comparing one measure with another (e.g., number of seizures compared with seizure duration). When significant differences were indicated by MANOV A or ANOVA analyses, Tukey's honestly significant difference post hoc tests were used to identify which effects were significant (p<0.05).

### Laboratory Example 1

### Control Experiments

In control experiments in which PTZ was administered, but no IO nerve stimulation was provided, the average number of seizures per minute was 5.98±0.45, and the average seizure duration was 3.94±0.23 sec. In contrast to studies of VNS in rats (Woodbury & Woodbury, (1990) Epilepsia 31 [Suppl. 2]: S7-S19) and dogs (Zabara, (1992) Epilepsia 33:1005-1012), we did not observe any substantial cardiovascular side effects during IO nerve stimulation (FIG. 2). Electrocardiogram (EKG) signals in anesthetized rats were recorded while stimulating the IO nerve and no substantial change in heart rate during stimulation was observed.

### Laboratory Example 2

### Stimulation of the lnfraorbital Nerve Reduces Seizure Activity

Stimulation of the intraorbital nerve by employing the periodic stimulation paradigm substantially reduces PTZ-induced seizure activity compared with that of control periods, as shown in FIGS. 3, 4 and 5. This effect is dependent on both the current and the frequency of the stimulation. There are no significant differences between the cortex and thalamus on any of the measures (Rao R(3,134)=0.33; p.>0.8].

The seizure reduction effect of IO nerve stimulation is greater with increasing current levels, as depicted in FIGS. 3B-3D. For the experiments described in FIGS. 3B-3D, pulse duration and frequency were held constant at 0.5 msec and 333 Hz, respectively, while current was varied between 3 and 11 mA, in 2 mA increments. At currents of 3 and 5 mA, there were no differences between periods of IO nerve stimulation and periods of no stimulation. However, at 7, 9 and 11 mA, nerve stimulation caused a significant decrease in overall seizure activity (as shown in FIG. 3B, 7 mA, 43.2±7.0%; 9 mA, 65.5±4.7%; 11 mA, 77.5±4.3%; p<0.001) and in the number of seizures initiated (as shown in FIG. 3C,7 mA,36.4±5.8%; 9 mA, 50.5±4.6%;11 mA, 58.7±6%; p<0.0001). There was also a significant decrease in the seizure duration at 9 mA (as shown in FIG. 3D, 52.5±3.7%; p<0.0001).

Different stimulus frequencies can have different effects on the seizure activity, as indicated by the data presented in FIG. 4. For example, in the experiments described in FIG. 4, pulse duration and current were held constant at 0.5 msec and 9 mA, respectively. Stimulation at high frequencies (100, 125, 200, and 333 Hz) caused a significantly smaller number of seizures than did periods of no stimulation, as disclosed in FIG. 4A (p<0.05) and as described above. However, stimulation frequencies of 50 Hz and lower did not cause any significant changes in the number of seizures initiated (FIG. 4A; p=1.0), but seizures did tend to be longer than those during control periods at these frequencies (FIG. 4B; 10 Hz; p<0.02).

### Laboratory Example 3

### Bilateral Versus Unilateral Stimulation

Bilateral stimulation is significantly more effective at reducing seizures than is unilateral stimulation either contralateral or ipsilateral to the recording site, as shown in FIG. 5. This effect is significant for the integrated seizure activity measure (FIG. 5B) at a current level of 7 mA (75.7±5.7%; p<0.002), as well as for the number of seizures (FIG. 5C) at 7 and 9 mA (7 mA, 63.7±5.3; 9 mA, 78.1±3.7%; p<0.01). A superior effect of bilateral stimulation is observed for the middle range of stimulation intensities that can be employed in accordance with the present invention. That is, if the current is too low, presumably below the threshold for seizure reduction, it is not necessary to stimulate both nerves. However, it was observed that if the current is high enough, stimulating unilaterally can be as effective as stimulating bilaterally. In the middle range of stimulation intensities, however, bilateral stimulation is permits the use of less current per nerve while still maintaining a high degree of seizure reduction.

### Laboratory Example 4

### Automatic Detection of Seizure Activity and Termination of Seizures

Employing the ASD device to stimulate the IO nerve only when seizure activity is detected reduces the amount of seizure activity. FIG. 6 shows that when the seizure detector identifies seizure activity in the field potential traces and triggers the stimulator, the seizure stops. The degree of seizure reduction can be dependent on the current level, which is represented in FIG. 7. For the experiments depicted in FIG. 7, the pulse duration was constant at 0.5 msec, and the frequency is constant at 333 Hz. Current was varied from 3 to 11 mA in 2 mA increments. FIG. 7B shows that the integrated seizure activity level is significantly reduced at 9 and 11 mA (9 mA, 55.2±7.2%; p <0.03; 11 mA, 56.6±8.0%; p<0.01). The number of seizures was significantly decreased at 7 and 9 mA, as shown in FIG. 7C, 7 mA, 19.3±5.8%; p<0.05; 9 mA, 22.5±6.1 %; p<0.0001). In addition, the seizure duration was decreased at 7,9 and 11 mA, as shown in FIG. 7D, 7 mA, 40.2±3.3%; 9 mA, 45.2±3.6%; 11 mA, 49.4±4.0%; p<0.0001 for all.

To compare the efficacy of the ASD with that of the periodic stimulation paradigm by calculating the ratio of the percent of seizure reduction to stimulus-on time was calculated, as shown in FIG. 8. By comparing these ratios between ASD stimulation and periodic stimulation protocols, it was determined that at least in the acute seizure model (PTZ) employed in the present experiment, delivering stimulation only when seizure activity was detected is up to 39.8 times more effective at seizure reduction per second of stimulation than is periodic stimulation not related in any way to seizure activity.

There was a difference between the nature of the seizure reduction effect obtained by employing the ASD device and that obtained using the periodic stimulation paradigm described hereinabove. When the periodic stimulation paradigm was employed, the number of seizures and the seizure durations were reduced by approximately the same amount at each current level (depicted in FIG. 3, and clear from a comparison of FIGS. 3C and 3D). However, as shown in FIG. 7 (compare FIGS. 7C and 7D; p<0.000001), when the ASD device is employed, the seizure durations are reduced significantly more than the number of seizures.

In addition, analysis of the data revealed that in control experiments where PTZ was administered but no stimulation was provided, the average time between the end of one spontaneously occurring seizure and the beginning of the next is 6.1 sec (calculated from the average number of seizures and the average seizure duration). The latency between the end of a stimulus and the next spontaneous seizure (i.e., in the epoch after a stimulus-on period) is observed to be 7.59±1.29 sec. Thus, the average delay between the end of a period of stimulation and the next spontaneously occurring seizure is an average of 24% longer than the interseizure interval during control experiments where no stimulation is present.

### Laboratory Example 5

### Automatic Detection of Seizure Activity and Termination of Seizures

Use of the ASD device to stimulate the IO nerve only when seizure activity was detected successfully reduced the amount of seizure activity relative to control periods. FIG. 6 demonstrates that when the seizure detector identified seizure activity in the field potential traces and triggered the stimulator, the seizure stopped. As in the experiments described above, the degree of seizure reduction was dependent on the current level (FIG. 7). For this set of experiments, the pulse duration was held constant at 0.5 msec, and the frequency at 333 Hz. Current was varied from 3 to 11 mA in 2 mA increments. FIG. 7B shows that the integrated seizure activity level was significantly reduced at 9 and 11 mA (9 mA, 55.2±7.2%;p<0.03; 11 mA, 56.6±8.0%; p<0.01). The number of seizures was significantly decreased at 7 and 9 mA (FIG. 7C, 7 mA, 19.3±5.8%; p<0.05; 9 mA, 22.5±6.1%;p<0.0001). In addition, the seizure duration was decreased at 7,9, and 11 mA (FIG. 7D, 7 mA, 40.2±3.3%; 9 mA, 45.2±3.6%; 11 mA, 49.4±4.0%;p<0.0001 for all). To compare the efficacy of the ASD device with that of the periodic stimulation paradigm, the ratio of the percent of seizure reduction to stimulus-on time was calculated (FIG. 8). By comparing these ratios between ASD stimulation and periodic stimulation protocols, it was observed that, at least in the acute seizure model (PTZ) used in these experiments, delivering stimulation only when seizure activity was detected was up to 39.8 times more effective at seizure reduction per second of stimulation than was periodic stimulation not related in any way to seizure activity.

There was an important difference between the nature of the seizure reduction effect using the ASD device and that observed using the periodic stimulation paradigm described above. With the periodic stimulation paradigm, the number of seizures and the seizure durations were reduced by approximately the same amount at each current level (FIG. 3, compare 3C and 3D). However, when the ASD device was used, the seizure durations were reduced significantly more than the number of seizures (FIG. 7, compare 7C and 7D; p<0.000001).

In addition, analysis of the data revealed that in control experiments where PTZ was administered but no stimulation was provided, the average time between the end of one spontaneously occurring seizure and the beginning of the next was 6.1 sec (calculated from the average number of seizures and the average seizure duration). We also measured the latency between the end of a stimulus and the next spontaneous seizure (i.e., in the epoch after a stimulus-on period), which was 7.59±1.29 sec. Thus, the average delay between the end of a period of stimulation and the next spontaneously occurring seizure is an average of 24% longer than the interseizure interval during control experiments where no stimulation was present.

### Discussion of Laboratory Examples 1 to 5

The following discussion refers the data and procedures described in Laboratory Examples 1 to 5.

### Mechanism of Seizure Reduction by Cranial Nerve Stimulation

While it is not the inventors' desire to be bound to any theory, it is postulated that the mechanism by which cranial nerve stimulation causes desynchronization of thalamic and cortical activity and reduces seizure is that such stimulation activates the midbrain reticular formation and that this activation results in generalized arousal via the reticular-activating system. In support of this view, it has been shown that stimulation of the midbrain reticular formation suppresses focal strychnine spikes in cats (Gellhorn, (1960) Electroencephalogr. Clin. Neurophysiol. 12: 613-19). In addition, several methods of eliminating seizure-related activity by activating multiple sensory modalities have been demonstrated. These include the reduction of absence seizures by acoustic stimuli (Raina & Lona, (1989) Epilepsia 30: 168-174) and the reduction of interictal focal activity or absence seizures by motor or mental activity (Jung, (1962) Epilepsia 3: 435-37; Ricci et al., (1972) Epilepsia 13: 785-94) or by thermal stimulation (McLachlan, (1993) Epilepsia 34: 918-23). Because such a wide range of manipulations can reduce seizure-related activity, it is reasonable to suggest that seizure reduction in these cases is caused by a generalized effect on arousal mediated by the brainstem reticular formation. This postulation is supported by the classical work of Moruzzi and Magoun (Moruzzi & Magoun, (1949) Electroencephalogr. Clin. Neurophysiol. 1: 455-73), demonstrating that stimulation of the midbrain reticular formation causes EEG desynchronization. This hypothesis is consistent with the observation disclosed in the context of the present invention that seizure reduction effects are not specific to the vagus nerve, but can instead be achieved by stimulation of multiple cranial nerves that convey information to the reticular formation.

One factor to consider with regard to both the mechanism of seizure reduction by trigeminal nerve stimulation and its applicability to long-term use in humans is the nature of the fiber types that are preferably activated to cause the seizure reduction effect. Multiple studies of the VNS technique have shown that the level of stimulation, in terms of stimulus frequency and intensity, must be high enough to activate slowly conducting c-fibers (Chase et al., (1967) Brain Res. 5: 236-249; Woodbury & Woodbury, (1990) Epilepsia 31[Suppl.2]: S7-S19). It is noted that the frequency range to be therapeutic in the present invention is somewhat different from that typically used in animal and human VNS studies. In animal studies the usual therapeutic range was generally 10-30 Hz (Woodbury & Woodbury, (1990) Epilepsia 31[Suppl.2]: S7-S19; Zabara, (1992) Epilepsia 33: 1005-1012; Takaya et al., (1996) Epilepsia 37: 1111-1116), although higher stimulation frequencies (50-250 Hz) were employed in monkeys (Lockard et al., (1990) Epilepsia 31[Suppl. 2]: S20-S26). In human studies the range used for stimulation was typically 20-30 Hz (McLachlan, (1997) J. Clin. Neurophysiol. 14: 358-68). This difference between VNS studies and the present disclosure might be caused by the difference in the relative numbers of fiber types between the vagus nerve and the infraorbital nerve. In cat, the vagus nerve is composed of 65-90% unmyelinated fibers (Foley & DuBois, (1937) J. Comp. Neurol 67:49-67; Agostoni et al., (1957) J. Physiol. (London) 135:182-205), whereas the rat IO nerve contains ˜33% slowly conducting, unmyelinated fibers (Klein et al., (1988) J. Comp. Neurol. 268: 469-488). However, it is not clear what the relationship is between fiber composition and the stimulus frequency/intensity required for seizure reduction, so interpreting these differences is non-trivial. A factor to consider is that although it has been shown that for seizure reduction the level of stimulation must be sufficient to activate c-fibers, these fibers might not be necessary for the seizure reduction effect. Finally, it is important to note that according to several studies (Torebjork, (1974) Acta Physiol. Scand. 92: 374-390; Torebjork & Hallin, (1974) J. Neurol. Neurosurg. Psychiatry 37: 653-664), c-fibers do not conduct if electrical stimuli are presented at frequencies above ˜10 Hz. This implies that although high stimulation frequencies are preferable to induce the seizure reduction effect of the present invention, it is possible that at such frequencies the c-fibers are not activated or are activated to a lesser degree than other fibers in the nerve.

Furthermore, it is possible that cells in the trigeminal nucleus are not able to follow with sustained responses at the high rates of stimulation provided in the present invention. For example, it has been demonstrated using a slice preparation of the rat medulla that neurons in the nucleus of the solitary tract (NTS) responded with lower EPSP amplitudes as the frequency of solitary tract stimulation was increased (Andresen & Yang, (1995) J. Neurophysiol. 74:1518-1528). These results might be relevant to the trigeminal nerve stimulation aspect of the present invention. This study also demonstrated that bursts of high-frequency stimulation resulted in less EPSP attenuation than did continuous high-frequency stimulation, suggesting that an optimal stimulation protocol could involve short bursts of high-frequency stimulation rather than continuous trains.

The delay between the onset of seizure-triggered stimulation and the end of the seizure activity might shed some light on the mechanism by which trigeminal stimulation reduces seizure activity. The average time between the onset of the seizure-triggered stimulus and the end of the seizure was 529.9±40.3 msec (note that FIG. 6 demonstrates some of the shortest delays). It is interesting that this value is similar to the minimum effective stimulus train duration (500 μsec) that was determined empirically. However, it should be noted that there was a wide range of delays, and this might be caused by at least two factors. First, the phase of the synchronous oscillations during which the stimuli occur might have an impact on the efficacy of the stimulation. Second, it is possible that the ability to abort a seizure varies depending on the amount of time the seizure has been ongoing before a sufficient stimulus arrives. Thus, differences in the phase of the oscillatory seizure activity at which the stimuli occur or the threshold used for seizure detection might affect the efficacy of the stimulation. These mechanisms can explain the variation in the amount of time required to abort a seizure.

Another important mechanism-related issue is whether the trigeminal stimulation was able to stop seizure activity during the stimulation itself or whether it also had an effect on the number of seizures initiated. In control files where PTZ was administered but no stimulation was provided, the average time between the end of one spontaneously occurring seizure and the beginning of the next was 6.1 sec (calculated from the average number of seizures and the average seizure duration). The latency between the end of a period of stimulation and the next spontaneous seizure (i.e., in the epoch after a stimulus-on period), was also measured and determined to be 7.59±1.29 sec. Thus, the average delay between the end of a period of stimulation and the first spontaneous seizure after the stimulus ends is, on average, 24% longer than the interseizure interval during control files with no stimulation present. These observations and interpretations are supported by other studies (Zabara, (1992) Epilepsia 33: 1005-1012; Takaya et al., (1996) Epilepsia 37: 1111-1116) indicating that the seizure reduction effect of vagus nerve stimulation can outlast the stimulus duration.

### Bilateral Versus Unilateral IO Nerve Stimulation

It is demonstrated that bilateral stimulation is more effective than unilateral stimulation in the middle of the therapeutic-current range. This disclosure has implications for how such stimulation could be used to most effectively reduce seizure activity. Specifically, because bilateral stimulation at 7 mA is just as effective as unilateral stimulation at 11 mA (FIG. 5), the use of bilateral nerve cuff electrodes can reduce the amount of current delivered to each nerve, while still maintaining the same seizure reduction effect as higher stimulation current at a single site. This is beneficial because it can reduce the potential for damage to nerve fibers at the stimulation site (Agnew et al., (1989) Ann. Biomed. Eng. 17: 39-60; Agnew & McCreery, (1990) Epilepsia 31 [Suppl. 2]:S27-S32), and it can reduce the intensity of any possible side effects associated with the stimulation. Bilateral stimulation is a further improvement over VNS, because the vagus nerve cannot be safely stimulated bilaterally without substantial risk of cardiovascular side effects (Schachter & Saper, (1998) Epilepsia 39: 677-686).

It is important to point out that the disclosure of the fact that bilateral stimulation of the IO nerve was more effective than unilateral stimulation is in contrast to two previous studies, which reported that bilateral stimulation of the vagus nerve was no more effective than unilateral stimulation (Chase et al., (1966) Exp. Neurol. 16: 36-49; Zabara, (1992) Epilepsia 33: 1005-1012). This discrepancy is likely either caused by differences in fiber composition between the vagus nerve and the IO nerve or caused by the fact that the stimulus parameters used in those studies were beyond those for which bilateral stimulation is superior to unilateral stimulation. Details about the stimulus parameters used for assessing the efficacy of bilateral stimulation in those two studies were not provided.

Another important point to consider is that we have tested the effect of bilateral stimulation with the PTZ seizure model, which involves generalized, tonic-clonic seizures (Fisher, (1989) Brain Res. Rev. 14: 245-278). Additional testing of the present invention with focal seizure models, such as localized application of alumina gel (Lockard et al., (1990) Epilepsia 31[Suppl. 2]:S20-S26) or penicillin (McLachlan, (1993) Epilepsia 34: 918-923) to the cortex, can demonstrate an advantage to bilateral stimulation in eliminating these types of seizures as well. Evidence to support an advantage in using bilateral stimulation to treat focal seizures is that, when employing the present invention, unilateral stimulation eliminated seizure activity in both hemispheres at the same time, suggesting that the effect of the stimulation is not restricted to one hemisphere. Such results have also been found for VNS in cats (Chase et al., (1966) Exp. Neurol. 16: 36-49), dogs (Zabara, (1992) Epilepsia 33:1005-1012), and humans (Henry et al., (1998) Epilepsia 39: 983-990; Henry et al., (1999) Neurol. 52: 1166-1173). These results indicate that because each nerve being stimulated can reduce seizures bilaterally, the effect of stimulating both nerves might be additive within a given hemisphere.

### REFERENCES

The references listed below as well as all references cited in the specification are incorporated herein by reference to the extent that they supplement, explain, provide a background for or teach methodology, techniques and/or compositions employed herein.

Agnew & McCreery, (1990) Epilepsia 31 [Suppl. 2]: S27-S32

Agnew et al., (1989) Ann. Biomed. Eng. 17: 39-60

Agostoni et al., (1957) J. Physiol. (London) 135: 182-205

Andresen & Yang, (1995) J. Neurophysiol. 74: 1518-1528

Ben-Menachem et al., (1994) Epilepsia 35: 616-626

Chase & Nakamura, (1968) Brain Res. 5: 236-249

Chase et al., (1966) Exp. Neurol. 16: 36-49

Chase et al., (1967) Brain Res. 5: 236-249

Fanselow & Nicolelis, (1999) J. Neurosci. 19: 7603-7616

Fisher, (1989) Brain Res. Rev. 14: 245-278

Foley & DuBois, (1937) J. Comp. Neurol. 67: 49-67

Gabor et al., (1996) Electroencephalogr. Clin. Neurophysiol. 99: 257-266

Gellhorn, (1960) Electroencephalogr. Clin. Neurophysiol. 12: 613-19

Henry et al., (1999) Neurology 52: 1166-1173

Henry et al., (1998) Epilepsia 39: 983-990

Henry et al., (1999) Neurol. 52: 1166-1173

Jung, (1962) Epilepsia 3: 435-37

Klein et al., (1988) J. Comp. Neurol. 268: 469-488

Le Van Quyen et al., (1999) NeuroReport 10: 2149-2155

Lockard et al., (1990) Epilepsia 31[Suppl 2]: S20-S26

Magnes et al., (1961) Arch. Ital. Biol. 99: 33-67

Martinerie et al., (1998) Nat. Med. 4: 1173-1176

McLachlan, (1993) Epilepsia 34: 918-923

McLachlan, (1997) J. Clin. Neurophysiol. 14: 358-368

McNamara, (1999) Nature 399[Suppl. 6738]: A15-A22

Moruzzi & Magoun, (1949) Electroencephalogr. Clin. Neurophysiol. 1: 455-473

Nicolelis et al., (1997) Neuron 18: 529-537

Paxinos & Watson, (1986) The Rat Brain, Ed. 2. New York: Academic, Harcourt, Brace and Jovanovich

Penry & Dean, (1990) Epilepsia 31[Suppl. 2]: S40-S43

Rajna & Lona, (1989) Epilepsia 30:168-174

Ramsay et al., (1994) First International Vagus Nerve Stimulation Study Group, Epilepsia 35: 627-636

Ricci et al., (1972) Epilepsia 13: 785-94

Schachter & Saper, (1998) Epilepsia 39: 677-686

Takaya et al., (1996) Epilepsia 37: 1111-1116

Terry et al., (1990) Epilepsia 31 [Suppl 2]: S33-S37

Torebjork & Hallin, (1974) J. Neurol. Neurosurg. Psychiatry 37: 653-664

Torebjork, (1974) Acta Physiol. Scand. 92: 374-390

Uthman et al., (1990) Epilepsia 31[Suppl. 2]: S44-S50

Uthman et al., (1993) Neurology 43: 1338-1345

Vagus Nerve Stimulation Study Group, (1995) Neurology 45: 224-230

Webber et al., (1996) Electroencephalogr. Clin. Neurophysiol. 98: 250-272

Woodbury & Woodbury, (1990) Epilepsia 31[Suppl. 2]: S7-S19

Zabara, (1985) Epilepsia 26: 518

Zabara, (1992) Epilepsia 33: 1005-1012

Zanchetti et al., (1952) Electroencephalogr. Clin. Neurophysiol. 4: 357-361

U.S. Pat. No. 6,016,449

U.S. Pat. No. 6,061,593

U.S. Pat. No. 5,540,734

U.S. Pat. No. 4,702,254

U.S. Pat. No. 4,867,164

U.S. Pat. No. 5,025,807

It will be understood that various details of the invention may be changed without departing from the scope of the invention. Furthermore, the foregoing description is for the purpose of illustration only, and not for the purpose of limitation—the invention being defined by the claims.

What is claimed is:

1. An intelligent brain pacemaker for a mammal having a cranial nerve not associated with an autonomic function comprising:
(a) one or more electrodes adapted to acquire field potential measurements indicative of a mammal's brain activity in real-time;
(b) a seizure detector adapted to detect seizure-related brain activity of a mammal in real-time, the seizure detector being electrically connected to the one or more electrodes;
(c) one or more nerve stimulators adapted to provide electrical stimulation to a mammal's cranial nerve not associated with an autonomic function, to terminate or ameliorate the seizure, the one or more nerve simulators being electrically connected to the seizure detector; and
(d) a power source for providing power to the intelligent brain pacemaker.

2. The apparatus of claim 1, wherein the one or more electrodes comprise one or more microwire arrays comprising TEFLON®-coated stainless steel wires.

3. The apparatus of claim 2, wherein the stainless steel wires are about 50 μm in diameter.

4. The apparatus of claim 2, wherein the one or more microwire arrays comprise 8 or more TEFLON®-coated stainless steel wires.

5. The apparatus of claim 2, wherein the one or more microwire arrays comprise a bundle of 8 or more TEFLON®-coated stainless steel microwires.

6. The apparatus of claim 1, wherein the one or more electrodes comprise one or more microwire arrays comprising TEFLON®-coated tungsten wires.

7. The apparatus of claim 6, wherein the tungsten wires are about 50 μm in diameter.

8. The apparatus of claim 6, wherein the one or more microwire arrays comprise 8 or more TEFLON® coated tungsten wires.

9. The apparatus of claim 6, wherein the one or more microwire arrays comprise a bundle of 8 or more TEFLON®-coated tungsten microwires.

10. The apparatus of claim 1, wherein the one or more electrodes are adapted to be affixed to an exterior surface of a subject's body.

11. The apparatus of claim 1, wherein each of the one or more electrodes is separately monitored.

12. The apparatus of claim 1, wherein the field potential measurements indicative of a subject's brain activity are acquired continuously.

13. The apparatus of claim 1, wherein the seizure-related brain activity of a subject is detected continuously.

14. The apparatus of claim 1, wherein the seizure detector is adapted to:
(a) determine if any field potential measurement matches a predetermined known pattern of epileptic brain activity;
(b) send a signal to a stimulator if a field potential measurement matches a predetermined known pattern of epileptic brain activity;
(c) continue sending a signal to the stimulator for as long as the a field potential matches a predetermined known pattern of
(d) epileptic brain activity; and
(d) stop sending a signal to the stimulator when field potential measurements do not match a predetermined known pattern of epileptic brain activity.

15. The apparatus of claim 14, wherein the predetermined known pattern of epileptic brain activity is indicative of seizure activity.

16. The apparatus of claim 14, wherein the predetermined known pattern of epileptic brain activity comprises a field potential surpassing a threshold voltage value.

17. The apparatus of claim 1, wherein the seizure detector comprises a seizure detection algorithm running on a computer microchip.

18. The apparatus of claim 17, wherein the seizure detector is disposed on a computer.

19. The apparatus of claim 1, wherein the one or more nerve stimulators comprise a nerve cuff electrode.

20. The nerve cuff electrode of claim 19, wherein the nerve cuff electrode comprises one or more bands comprising a conductive material, every band being in electrical connection with every other band.

21. The apparatus of claim 1, wherein the one or more nerve stimulators comprise a device adapted to provide electrical stimulation when triggered.

22. The apparatus of claim 1, wherein the electrical stimulation is provided in the form of a pulse train.

23. The apparatus of claim 1, wherein the cranial nerve not associated with an autonomic function is a trigeminal nerve.

24. The apparatus of claim 23, wherein a branch of the trigeminal nerve is electrically stimulated.

25. The apparatus of claim 1, wherein the power source is a lithium battery.

26. The apparatus of claim 1, wherein the apparatus is adapted to be implanted in the brain tissue of a subject.

27. The apparatus of claim 1, wherein the apparatus is adapted to be implanted in the body of a subject.

28. The apparatus of claim 1, further comprising an operatively connected computer adapted to provide a visualization of the field potential data.

29. The apparatus of claim 28, wherein the computer is a handheld computer.

30. The apparatus of claim 1, further comprising circuitry adapted to transmit information by radio telemetry.

31. The apparatus of claim 30, wherein the information transmitted is selected from the group consisting of field potential information and seizure-related information.

32. A method of detecting and ameliorating a seizure, in a mammal having a cranial nerve not associated with an autonomic function, the method comprising:
(a) acquiring field potential data indicative of a subject's electrical brain activity in real-time;
(b) analyzing the field potential data to identify seizure-related brain activity;
(c) electrically stimulating a cranial nerve not associated with an autonomic function of the subject, if seizure-related brain activity is identified; and
(d) removing the stimulation when seizure-related brain activity is not detected, whereby a seizure is detected and ameliorated.

33. The method of claim 32, wherein the method is performed without generating a detectable cardiovascular side effect.

34. The method of claim 32, wherein the field potential data is acquired continuously.

35. The method of claim 32, wherein the field potential data is acquired via one or more microelectrode arrays comprising a plurality of microwires.

36. The method of claim 35, wherein the field potential data from each microwire of the microwire array is separately recorded.

37. The method of claim 35, wherein the one or more microwire arrays comprises one or more bundles of TEFLON®-coated stainless steel microwires.

38. The method of claim 35, wherein the one or more microwire arrays comprises one or more bundles of TEFLON®-coated tungsten microwires.

39. The method of claim 32, wherein the field potential data is acquired at a sampling rate of between 128 and 1024 Hz.

40. The method of claim 39, wherein the field potential data is acquired at a sampling rate of between 500 and 1024 Hz.

41. The method of claim 32, wherein the analyzing comprises the steps of:
(a) band-pass filtering the acquired field potential data;
(b) comparing the field potential data to a predetermined known pattern of epileptic brain activity; and
(c) determining if any of the field potential data exceeds the predetermined known pattern of epileptic brain activity.

42. The method of claim 41, wherein the band-pass filtering is at a frequency between 1 and 100 Hz.

43. The method of claim 41, wherein the band-pass filtering is at a frequency of 30 Hz.

44. The method of claim 41, wherein the band-pass filtering comprises employing a notch filter at 60 Hz.

45. The method of claim 41, wherein the predetermined known pattern of epileptic brain activity is indicative of one of: seizure-related brain activity and brain activity predictive of oncoming seizure activity.

46. The method of claim 32, wherein the electrical stimulation is automatically triggered if seizure-related brain activity is identified.

47. The method of claim 32, wherein the electrical stimulation is stopped when seizure-related brain activity is absent.

48. The method of claim 32, wherein the electrical stimulation of the cranial nerve not associated with an autonomic function is delivered by a nerve cuff electrode.

49. The method of claim 48, wherein the nerve cuff electrode comprises one or more conducting bands.

50. The method of claim 48, wherein the electrical stimulation is delivered as a train of electrical pulses.

51. The method of claim 50, wherein the train of electrical pulses comprises a 0.5 second train of 500 μs pulses at a frequency value of between 1 and 333 Hz.

52. The method of claim 50, wherein the train of electrical pulses comprises a 0.5 second train of 500 μs pulses at a current value of between 3 and 11 mA.

53. The method of claim 32, wherein the cranial nerve not associated with an autonomic function is a trigeminal nerve.

54. The method of claim 53, wherein a branch of the trigeminal nerve is electrically stimulated.

55. The method of claim 54, wherein a branch of the trigeminal nerve is unilaterally electrically stimulated.

56. The method of claim 54, wherein a branch of the trigeminal nerve is bilaterally electrically stimulated.

57. The method of claim 32, wherein the cranial nerve not associated with an autonomic function and one or more locations on a subject's body distinct from the cranial nerve not associated with an autonomic function are electrically stimulated.

58. The method of claim 57, wherein the one or more locations is selected from the group consisting of cranial nerves and brain tissue.

59. The method of claim 32, wherein steps (a) through (d) are repeated continuously.

60. The method of claim 32, wherein steps (a) through (d) are performed within the body of a subject.

61. A method of increasing the time between epileptic seizures, the method comprising:
(a) acquiring field potential data from the brain of a subject;
(b) analyzing the field potential data to identify the presence of an epileptic seizure in a subject;
(c) electrically stimulating a cranial nerve not associated with an autonomic function of the subject when an epileptic seizure is identified; and
(d) repeating steps (a) through (c), whereby the time between seizures is increased.

62. The method of claim 61, wherein the method is performed without generating a detectable cardiovascular side effect.

63. The method of claim 61, wherein the field potential data is acquired continuously.

64. The method of claim 61, wherein the field potential data is acquired from one or more microwire arrays.

65. The method of claim 64, wherein the field potential data from each microwire of the microwire array is separately recorded.

66. The method of claim 64, wherein the one or more microwire arrays comprises one or more bundles of TEFLON®-coated stainless steel microwires.

67. The method of claim 64, wherein the one or more microwire arrays comprises one or more bundles of TEFLON®-coated tungsten microwires.

68. The method of claim 61, wherein the field potential data is acquired at a sampling rate of between 128 and 1024 Hz.

69. The method of claim 68, wherein the field potential data is acquired at a sampling rate of between 500 and 1024 Hz.

70. The method of claim 61, wherein the analyzing comprises the steps of:
(a) band-pass filtering the acquired field potential data;
(b) comparing the field potential data to a predetermined known pattern of epileptic brain activity; and
(c) determining if any of the field potential data matches the predetermined known pattern of epileptic brain activity.

71. The method of claim 70, wherein the band-pass filtering is at a frequency between 1 and 100 Hz.

72. The method of claim 71, wherein the band-pass filtering is at a frequency of 30 Hz.

73. The method of claim 70, wherein the band-pass filtering comprises a notch filter at 60 Hz.

74. The method of claim 70, wherein the predetermined known pattern of epileptic brain activity is indicative of one of: seizure-related brain activity and brain activity predictive of oncoming seizure activity.

75. The method of claim 70, wherein the predetermined known pattern of epileptic brain activity comprises a field potential that exceeds a predetermined threshold value.

76. The method of claim 61, wherein the electrical stimulation is automatically triggered if seizure-related brain activity is identified.

77. The method of claim 61, wherein the electrical stimulation of the trigeminal nerve is delivered by a nerve cuff electrode.

78. The method of claim 77, wherein the nerve cuff electrode comprises a plurality of conducting bands.

79. The method of claim 61, wherein the electrical stimulation is delivered as a train of electrical pulses.

80. The method of claim 79, wherein the train of electrical pulses comprises a 0.5 second train of 500 μs pulses at a frequency value of between 1 and 333 Hz.

81. The method of claim 79, wherein the train of electrical pulses comprises a 0.5 second train of 500 μs pulses at a current value of between 3 and 11 mA.

82. The method of claim 61, wherein the cranial nerve not associated with an autonomic function and one or more distinct locations on a subject's body are electrically stimulated.

83. The method of claim 82, wherein the one or more distinct locations is selected from the group consisting of cranial nerves and brain tissue.

84. The method of claim 61, wherein the cranial nerve not associated with an autonomic function is a trigeminal nerve.

85. The method of claim 61 or 84, wherein the cranial nerve not associated with an autonomic function is unilaterally electrically stimulated.

86. The method of claim 61 or 84, wherein the cranial nerve not associated with an autonomic function is bilaterally electrically stimulated.

87. The method of claim 61, wherein steps (a) through (d) are performed within the body of a subject.
